(19) United States
(12) Patent Application Publication
(10) Pub. No.: US 2026/0158097 A1
(43) Pub. Date: Jun. 11, 2026

(54) ANTI-BACTERIAL COMPOSITION COMPRISING EXTRACT OF COFFEE COMPOSITION AND ANTIBIOTIC, AND METHOD FOR TREATING BACTERIAL INFECTION BY USING THE SAME

(71) Applicants: KING'S GROUP BIOTECH CO., LTD, Pingtung County (TW); NATIONAL CHIAYI UNIVERSITY, Chiayi City (TW)

(72) Inventors: Chia-Chen PI, Pingtung County (TW); Chun-Chia CHEN, Pingtung County (TW); Jia-Shiun LI, Pingtung County (TW); Yi-Chen LIN, Pingtung County (TW); Yu-Chieh CHENG, Pingtung County (TW); Hung-Chih KUO, Chiayi City (TW); Kun-Wei CHAN, Chiayi City (TW); Ching-Fen WU, Chiayi City (TW)

(21) Appl. No.: 19/410,014

(22) Filed: Dec. 5, 2025

Related U.S. Application Data
(60) Provisional application No. 63/730,300, filed on Dec. 10, 2024.

(51) Int. Cl. A61K 36/74; A61K 31/165; A61K 31/424; A61K 31/43; A61K 31/545; A61K 31/65; A61K 31/7036; A61P 31/04

(52) U.S. Cl. CPC A61K 36/742; A61K 31/165; A61K 31/424; A61K 31/43; A61K 31/545; A61K 31/65; A61K 31/7036; A61P 31/04; A61K 2236/15; A61K 2236/17; A61K 2236/19; A61K 2236/333

(57) ABSTRACT

Provided is an anti-bacterial composition and a use thereof, specifically involving an anti-bacterial composition comprising an extract of a coffee composition and an antibiotic, and a use of the anti-bacterial composition for manufacturing an anti-bacterial medicament. The coffee composition comprises a solid content and water. The solid content comprises coffee beans and an auxiliary material. The antibiotic is selected from penicillins, cephalosporins, fluoroquinolones, tetracyclines, chloramphenicols, aminoglycosides, and a combination thereof. The ratio of the coffee composition to the antibiotic is in an amount ranging from 1.5:1 to 25000:1. The anti-bacterial composition has a better anti-bacterial effect than the antibiotic alone, and the anti-bacterial effect may also be effective against drug-resistant bacteria.

### CROSS-REFERENCE TO RELATED APPLICATION

Pursuant to 35 U.S.C. § 119(e), this application claims the benefit of the priority to U.S. Provisional Patent Application No. 63/730,300, filed on Dec. 10, 2024. The content of the prior application is incorporated herein by its entirety.

### BACKGROUND OF THE INVENTION

### 1. Field of the Invention

The present invention is related to an anti-bacterial composition, wherein the anti-bacterial composition comprises an extract of a coffee composition and an antibiotic. Besides, the present invention is related to a method for treating bacterial infection by using the anti-bacterial composition.

### 2. Description of the Prior Arts

To maintain the health and promote the growth of economic animals, such as poultry, livestock, and aquatic animals, producers often use anti-bacterial medicaments, such as antibiotic growth promoters (AGPs), to prevent the spread of diseases among animals. In environments with high stocking density or poor sanitary conditions, AGPs can effectively reduce animal morbidity and mortality, thereby ensuring production efficiency and profitability. Removing the use of AGPs, however, may result in production losses. For instance, discontinuing AGP use in post-weaning pigs can increase the feed cost per pig by US$0.86, and discontinuing AGP use during the growing and finishing stages can increase the feed cost per pig by US$3.11 (Cardinal et al., 2021).

However, the long-term or excessive use of anti-bacterial medicaments can lead to the emergence of drug-resistant bacteria, thereby compromising the effectiveness of the drugs. Producers frequently add low-dose antibiotics into feed or animal farming water to achieve a preventative effect, causing bacteria within the animals' bodies to develop resistance to the anti-bacterial medicaments, so the once-effective medications are unable to work. With the understanding of antibiotic abuse and related health risks, and with the increasing global calls to reduce the use of anti-bacterial medicaments in animal husbandry, many countries and consumers are seeking more sustainable farming practices to replace traditional antibiotic use models.

A study focusing on low- and middle-income countries (Boeckel et al., 2019) examined microbial drug resistance in economic animals like chickens, pigs, and cattle between 2000 and 2018. The study found a marked increase in the number of drug-resistant strains in chickens and pigs, with the proportion of anti-bacterial medicaments having over 50% resistance rising two to three times. Drug resistance affects not only economic animals but also farmers (Bogaard et al., 2001). Farmers who raise broilers and turkeys tend to use antibiotics more frequently than laying-hen farmers, so that E. coli with higher antibiotic resistance is found in the defecation of turkey and broiler farmers and slaughterhouse workers compared to laying-hen farmers, indicating that the transmission of E. coli resistance vectors from poultry to humans is quite common.

According to a report published by the U.S. Food and Drug Administration (FDA) in 2022, the use of anti-bacterial medicaments in broilers continuously declined between 2016 and 2022, and the use of anti-bacterial medicaments in beef cattle and pork pigs was also lower from 2017 to 2022 compared to 2016. This shows a trend of actively controlling the use of antimicrobial drugs. According to data from the European Surveillance of Veterinary Antimicrobial Consumption (ESVAC) program, among antibiotics for animals in 25 European countries, there was a significant decrease in the sales of Polymyxins and Fluoroquinolones between 2011 and 2018, showing a shift in the European animal antibiotic market.

Consequently, there is an urgent need to develop and seek out new products with anti-bacterial effects or alternative solutions to reduce reliance on traditional antibiotics, thereby solving the difficulties in managing animal diseases caused by restricted antimicrobial use, and the health of animals and the sustainable development of the farming industry can thus be guaranteed.

### SUMMARY OF THE INVENTION

Based on the needs mentioned above, one objective of the present invention is to provide a composition having an anti-bacterial effect. Compared to the use of traditional antibiotics, the composition can inhibit bacterial growth more effectively, reduce antibiotic dosage, and defend from drug-resistant bacteria.

Another objective of the present invention is to provide a method for treating bacterial infection.

Another objective of the present invention is to provide a use of the anti-bacterial composition.

To achieve the aforementioned objective, the present invention provides an anti-bacterial composition comprising:
an extract of a coffee composition, in which the coffee composition comprises 92% by weight (wt %) to 95 wt % of a solid content and 5 wt % to 8 wt % of water, the solid content comprises 20 wt % to 70 wt % of coffee beans, and 30 wt % to 80 wt % of an auxiliary material; and the coffee composition has a carbon-nitrogen ratio ranging from 35 to 50; and
an antibiotic selected from penicillins, cephalosporins, fluoroquinolones, tetracyclines, chloramphenicols, aminoglycosides, and a combination thereof;
wherein, the ratio of the coffee composition to the antibiotic is in an amount of 1.5:1 to 25000:1; and the extract of the coffee composition comprises a water extract of the coffee composition, an alcohol extract of the coffee composition, or a combination thereof.

The present invention further provides a method for treating bacterial infection by using the anti-bacterial composition. The method comprises administering the anti-bacterial composition to an animal in need to inhibit bacterial infection. In some embodiments, the method comprises administering a therapeutic effective amount of the anti-bacterial composition to an animal in need.

The present invention further provides a use of the anti-bacterial composition for manufacturing an anti-bacterial medicament.

When the coffee composition is extracted with the alcohol or water and incorporated into the anti-bacterial composition of the present invention, the coffee composition can form an anti-bacterial synergistic effect with the antibiotic, allowing the anti-bacterial composition of the present invention to exhibit a better anti-bacterial effect than using the antibiotic alone. Furthermore, the anti-bacterial effect is also active against drug-resistant bacteria, which overcomes the problem of decreased anti-bacterial ability associated with the increasing frequency of use of traditional anti-bacterial medicaments.

In the present invention, the carbon-nitrogen ratio of the coffee composition refers to a ratio of mass of carbon to mass of nitrogen in the coffee composition. In some embodiments, the coffee beans are raw coffee beans, roasted coffee beans, or recycled coffee grounds, wherein the raw coffee beans and the roasted coffee beans comprise a whole coffee bean, a coffee bean grit, a coffee powder, or a combination thereof.

In some embodiments, the coffee composition comprises the alcohol extract of the coffee composition.

In some embodiments, the coffee beans may have a particle size of 500 micrometers (μm) to 8300 μm.

In some embodiments, the recycled coffee grounds may have a particle size of 500 μm to 2000 μm. In some embodiments, the whole coffee bean may have a particle size of 5000 μm to 8300 μm, the coffee bean grit may have a particle size of 1000 μm to 5000 μm, and the coffee powder may have a particle size of 500 μm to 1000 μm.

In some embodiments, the coffee composition is a ground coffee composition with a particle size of 8300 μm or less. In other embodiments, the ground coffee composition has a particle size of 500 μm to 8300 μm, 500 μm to 8000 μm, 500 μm to 800 μm, 1000 μm to 2000 μm, 2000 μm to 3000 μm, 3000 μm to 4000 μm, 4000 μm to 5000 μm, 300 μm to 2000 μm, 500 μm to 1000 μm, 200 μm to 1000 μm, 100 μm to 830 μm, or 1000 μm to 7500 μm.

In some embodiments, the ratio of the coffee composition to the antibiotic is in an amount of 6.25:1 to 2500:1, 12.5:1 to 1250:1, 25:1 to 625:1, or 50:1 to 250:1.

In some embodiments, the alcohol extract is prepared by extracting the coffee composition with an ethanol aqueous solution having a concentration of 60% to 80%, wherein the ratio of the coffee composition to the ethanol aqueous solution is from 1 gram (g) to 3 milliliters (mL) to 1 g to 7 mL. In other embodiments, the ratio of the coffee composition to the ethanol aqueous solution is from 1 g to 4 mL to 1 g to 6 mL, or 1 g to 5 mL.

In some embodiments, the auxiliary material comprises a corn grit, a beet grit, a rice husk, a shelled soybean meal, a broken rice, or a combination thereof.

In some embodiments, the coffee composition is unfermented or fermented by Aspergillus oryzae.

According to the present invention, the coffee composition is prepared by the following processes: (1) mixing 40 wt % to 60 wt % of the solid content and 40 wt % to 60 wt % of water to obtain a mixture; (2) heating the mixture at 115° C. to 125° C. under a pressure of 1.0 bar to 1.5 bars for 20 minutes to 60 minutes to obtain an autoclaved mixture; and (3) cooling and drying the autoclaved mixture until the water content of the autoclaved mixture is reduced to 5 wt % to 8 wt % to obtain the coffee composition.

In some embodiments, step (3) comprises: cooling the autoclaved mixture, inoculating Aspergillus oryzae in the coffee composition with 1×10⁷ to 1×10⁸ spores per gram of coffee composition, and fermenting at 25° C. to 35° C. for 3 days to 7 days; and drying the autoclaved mixture until the water content of the autoclaved mixture is reduced to 5 wt % to 8 wt % to obtain the coffee composition.

In some embodiments, the penicillin is selected from penicillin G, penicillin V, ampicillin, amoxicillin, and amoxicillin-clavulanic acid.

In some embodiments, the cephalosporin is selected from cephalexin, cephadroxil, and ceftiofur.

In some embodiments, the fluoroquinolone is selected from enrofloxacin, ofloxacin, danofloxacin, ciprofloxacin, and norfloxacin.

In some embodiments, the tetracycline is selected from oxytetracycline, chlortetracycline, and doxycycline.

In some embodiments, the chloramphenicol is selected from chloramphenicol, thiamphenicol, and florfenicol.

In some embodiments, the aminoglycoside is selected from streptomycin, neomycin, kanamycin, gentamicin, spectinomycin, and apramycin.

According to the present invention, the anti-bacterial composition has bacterial inhibition effect on a bacterium selected from Salmonella spp., Escherichia spp., Pasteurella spp., Streptococcus spp., Gallibacterium spp., Staphylococcus spp., Riemerella spp., Pseudomonas spp., and Klebsiella spp.

In some embodiments, the bacterium is selected from Salmonella enterica, Salmonella choleraesuis, Escherichia coli, Pasteurella multocida, Glaesserella parasuis, Streptococcus suis, Gallibacterium anatis, Staphylococcus hyicus, Riemerella anatipestifer, Pseudomonas aeruginosa, and Klebsiella pneumoniae.

In some embodiments, the bacterium is a drug-resistant bacterium.

According to the present invention, the coffee composition can relieve the inflammation symptoms caused by lipopolysaccharide (LPS). The inflammation symptoms include the production of proteins such as interleukin-6 (IL-6) and tumor necrosis factor alpha (TNF-α) in cells and the production of nitric oxide (NO) in macrophages.

In some embodiments, the animal may be a livestock, a poultry, or an aquaculture animal, wherein the livestock comprises pig, cattle, cow, sheep, or goat, the poultry comprises chicken, duck, or goose, and the aquaculture animal comprises fish or shrimp.

### BRIEF DESCRIPTION OF THE DRAWINGS

FIG. 1A is an image of Escherichia coli colonies after culturing with florfenicol in each group in Test Example 1;

FIG. 1B is an image of Escherichia coli colonies after culturing with doxycycline in each group in Test Example 1;

FIG. 2A is a bar chart of colony forming units of Escherichia coli after culturing with florfenicol in each group in Test Example 1;

FIG. 2B is a bar chart of colony forming units of Escherichia coli after culturing with doxycycline in each group in Test Example 1;

FIG. 3A is an image of Escherichia coli colonies after culturing with cephalexin in each group in Test Example 2;

FIG. 3B is an image of Escherichia coli colonies after culturing with amoxicillin in each group in Test Example 2;

FIG. 3C is an image of Escherichia coli colonies after culturing with oxytetracycline in each group in Test Example 2;

FIG. 3D is an image of Escherichia coli colonies after culturing with doxycycline in each group in Test Example 2;

FIG. 3E is an image of Escherichia coli colonies after culturing with florfenicol in each group in Test Example 2;

FIG. 3F is an image of Escherichia coli colonies after culturing with gentamicin in each group in Test Example 2;

FIG. 3G is an image of Salmonella enterica colonies after culturing with cephalexin in each group in Test Example 2;

FIG. 3H is an image of Salmonella enterica colonies after culturing with amoxicillin in each group in Test Example 2;

FIG. 3I is an image of Salmonella enterica colonies after culturing with oxytetracycline in each group in Test Example 2;

FIG. 3J is an image of Salmonella enterica colonies after culturing with doxycycline in each group in Test Example 2;

FIG. 3K is an image of Salmonella enterica colonies after culturing with florfenicol in each group in Test Example 2;

FIG. 3L is an image of Salmonella enterica colonies after culturing with gentamicin in each group in Test Example 2;

FIG. 4A is a bar chart of colony forming units of Escherichia coli after culturing with cephalexin in each group in Test Example 2;

FIG. 4B is a bar chart of colony forming units of Escherichia coli after culturing with amoxicillin in each group in Test Example 2;

FIG. 4C is a bar chart of colony forming units of Escherichia coli after culturing with oxytetracycline in each group in Test Example 2;

FIG. 4D is a bar chart of colony forming units of Escherichia coli after culturing with doxycycline in each group in Test Example 2;

FIG. 4E is a bar chart of colony forming units of Escherichia coli after culturing with florfenicol in each group in Test Example 2;

FIG. 4F is a bar chart of colony forming units of Escherichia coli after culturing with gentamicin in each group in Test Example 2;

FIG. 5A is a bar chart of colony forming units of Salmonella enterica after culturing with cephalexin in each group in Test Example 2;

FIG. 5B is a bar chart of colony forming units of Salmonella enterica after culturing with amoxicillin in each group in Test Example 2;

FIG. 5C is a bar chart of colony forming units of Salmonella enterica after culturing with oxytetracycline in each group in Test Example 2;

FIG. 5D is a bar chart of colony forming units of Salmonella enterica after culturing with doxycycline in each group in Test Example 2;

FIG. 5E is a bar chart of colony forming units of Salmonella enterica after culturing with florfenicol in each group in Test Example 2;

FIG. 5F is a bar chart of colony forming units of Salmonella enterica after culturing with gentamicin in each group in Test Example 2;

FIG. 6 is an image of Escherichia coli colonies in each group in Test Example 3;

FIG. 7 is an image of Escherichia coli colonies in each group in Test Example 4;

FIG. 8A is a bar chart of colony forming units of Escherichia coli after culturing with cephalexin in each group in Test Example 4;

FIG. 8B is a bar chart of colony forming units of Escherichia coli after culturing with florfenicol in each group in Test Example 4;

FIG. 8C is a bar chart of colony forming units of Escherichia coli after culturing with doxycycline in each group in Test Example 4;

FIG. 9A is a bar chart of MIC90 of porcine Escherichia coli after culturing with florfenicol in each group in Test Example 5;

FIG. 9B is a bar chart of MIC90 of porcine Escherichia coli after culturing with doxycycline in each group in Test Example 5;

FIG. 10A is a bar chart of MIC90 of chicken Escherichia coli after culturing with florfenicol in each group in Test Example 5;

FIG. 10B is a bar chart of MIC90 of chicken Escherichia coli after culturing with enrofloxacin in each group in Test Example 5;

FIG. 10C is a bar chart of MIC90 of chicken Escherichia coli after culturing with doxycycline in each group in Test Example 5;

FIG. 11A is a bar chart of MIC90 of porcine Salmonella enterica after culturing with florfenicol in each group in Test Example 5;

FIG. 11B is a bar chart of MIC90 of porcine Salmonella enterica after culturing with enrofloxacin in each group in Test Example 5;

FIG. 11C is a bar chart of MIC90 of porcine Salmonella enterica after culturing with doxycycline in each group in Test Example 5;

FIG. 12A is a bar chart of MIC90 of chicken Salmonella enterica after culturing with florfenicol in each group in Test Example 5;

FIG. 12B is a bar chart of MIC90 of chicken Salmonella enterica after culturing with enrofloxacin in each group in Test Example 5;

FIG. 12C is a bar chart of MIC90 of chicken Salmonella enterica after culturing with doxycycline in each group in Test Example 5;

FIG. 13A is a bar chart of MIC90 of Pasteurella multocid after culturing with florfenicol in each group in Test Example 5;

FIG. 13B is a bar chart of MIC90 of Pasteurella multocid after culturing with doxycycline in each group in Test Example 5;

FIG. 14A is a bar chart of MIC90 of Glaesserella parasuis after culturing with florfenicol in each group in Test Example 5;

FIG. 14B is a bar chart of MIC90 of Glaesserella parasuis after culturing with enrofloxacin in each group in Test Example 5;

FIG. 14C is a bar chart of MIC90 of Glaesserella parasuis after culturing with doxycycline in each group in Test Example 5;

FIG. 15A is a bar chart of colony forming units of Escherichia coli in each group in Test Example 6;

FIG. 15B is a bar chart of colony forming units of Salmonella enterica in each group in Test Example 6;

FIG. 15C is a bar chart of colony forming units of Lactobacillus pentosus in each group in Test Example 6;

FIG. 15D is a bar chart of colony forming units of Lactobacillus reuteri in each group in Test Example 6;

FIG. 16A is a bar chart of absorbance values at 625 nm in each group in Test Example 7;

FIG. 16B is a bar chart of absorbance values at 260 nm in each group in Test Example 7;

FIG. 16C is a bar chart of protein concentrations in each group in Test Example 7;

FIG. 16D is a bar chart of absorbance values at 420 nm in each group in Test Example 7;

FIG. 17A shows images of Escherichia coli in each group taken by a scanning electron microscope in Test Example 8;

FIG. 17B shows images of Salmonella enterica in each group taken by a scanning electron microscope in Test Example 8;

FIG. 18A is a gel image of Escherichia coli for DNA damage assay in Test Example 9;

FIG. 18B is a gel image of Salmonella enterica for DNA damage assay in Test Example 9;

FIG. 18C is a gel image of test groups 9-4 to 9-9 in protein damage assay in Test Example 9;

FIG. 19A is a bar chart of colony forming units of Escherichia coli in each group in Test Example 10;

FIG. 19B is a bar chart of colony forming units of Escherichia coli in each group in Test Example 10;

FIG. 20A is a bar chart of Interleukin-6 concentrations in each group in Test Example 11;

FIG. 20B is a bar chart of nitric oxide yields in each group in Test Example 11;

FIG. 20C is a bar chart of IL-6 concentrations in each group in Test Example 11;

FIG. 20D shows images in each group taken by a scanning electron microscope in Test Example 11; and

FIG. 21 is a bar chart of nitric oxide yields in each group in Test Example 12.

### DETAILED DESCRIPTION OF THE PREFERRED EMBODIMENTS

Several examples are listed below with figures for further illustration. A person skilled in the art can easily realize the advantages and effects of the present invention from the following examples. The listed examples of the present invention are just for demonstration of implementation but not intended to limit the scope of the present invention. Various modifications and variations could be made to practice or apply the present invention without departing from the spirit and scope of the present invention.

### Preparation Example 1: An Extract of a Fermented Coffee Composition Prepared by Raw Coffee Beans or Roasted Coffee Beans

### (a) Mixing of Raw Materials

20 wt % to 40 wt % of raw coffee beans or roasted coffee beans, 40 wt % to 60 wt % of a corn grit, and 20 wt % to 40 wt % of a beet grit were mixed to obtain a first solid content having a carbon-nitrogen ratio ranging from 35 to 50; and water was added into the first solid content to obtain a first mixture with a water content of 40 wt % to 60 wt %. Herein, the raw coffee beans and the roasted coffee beans were a whole coffee bean, a coffee bean grit, a coffee powder, or a combination thereof. The grit refers to a granulated product with various particle sizes crushed from raw materials (such as coffee bean, corn, or beet).

### (b) Sterilizing and Fermenting

2 kilograms (kg) to 3 kg of the first mixture was placed into a fermentation vat and sterilized at 121° C. under a pressure of 1.2 bars for 30 minutes to obtain a first autoclaved mixture. The first autoclaved mixture was cooled to room temperature, and 1.5 g to 3 g of an Aspergillus oryzae powder (6.5×10⁷ spores per gram) (a strain selected by King's Ground Biotech, Aspergillus oryzae, FG003, identified by the Food Industry Research and Development Institute) was weighed and added into the first autoclaved mixture and fermented at 25° C. to 35° C. for 3 days to 7 days to obtain a first product. The first product was dried at 40° C. to 70° C. for 12 hours to 14 hours until the water content of the first product was reduced to 5 wt % to 8 wt %, and the dried first product was ground and filtered using a sieve with a pore size of 830 μm to obtain a fermented coffee composition of Preparation Example 1.

### (c) Extracting

5 g of the fermented coffee composition was placed into a 50 mL centrifuge tube and added with 25 mL of 70% ethanol or water to form a dispersion with a concentration of 200 milligrams (mg)/mL. The solution was shaken and mixed using a vortex mixer, and the centrifuge tube was placed in an incubator at a constant temperature of 30° C. and shaken at a speed of 120 revolutions per minute (rpm) for 2 hours. The centrifuge tube was taken out and centrifuged at a speed of 10,000 rpm for 10 minutes to collect a first supernatant. The first supernatant was sterilized at 121° C. for 20 minutes and centrifuged at a speed of 10,000 rpm for 10 minutes to collect a second supernatant. The second supernatant was filtered using a filter with a pore size of 0.22 μm to obtain an extract of Preparation Example 1. Herein, the extract was a water extract and/or an ethanol extract.

### Preparation Example 2: An Extract of a Fermented Coffee Composition Prepared by Recycled Coffee Grounds

The method of Preparation Example 2 to obtain the extract is similar to Preparation Example 1, wherein the differences include: in step (a) of Preparation Example 2, 40 wt % to 70 wt % of recycled coffee grounds and 30 wt % to 60 wt % of a corn grit were mixed to obtain a second solid content having a carbon-nitrogen ratio ranging from 35 to 50; and water was added into the second solid content to obtain a second mixture with a water content of 40 wt % to 60 wt %. An extract of Preparation Example 2 was obtained through the above steps, wherein the extract was a water extract and/or an ethanol extract.

### Preparation Example 3: An Extract of an Unfermented Coffee Composition Prepared by Roasted Coffee Beans

The method of Preparation Example 3 to obtain the extract is similar to Preparation Example 1, wherein the differences include: in step (a) of Preparation Example 3, 20 wt % to 40 wt % of roasted coffee beans, 40 wt % to 60 wt % of a corn grit, and 20 wt % to 40 wt % of a beet grit were mixed to obtain a third solid content having a carbon-nitrogen ratio ranging from 35 to 50; and water was added into the third solid content to obtain a third mixture with a water content of 40 wt % to 60 wt %. Fermenting step was omitted in step (b), that is, the third mixture was sterilized, cooled, and directly placed at 25° C. to 35° C. for 3 days to 7 days, followed by the subsequent steps of drying and grinding. An extract of Preparation Example 3 was obtained through the above steps, wherein the extract was a water extract and/or an ethanol extract.

The “extract concentration” used below refers to the milligrams of the coffee composition (unfermented or fermented) per milliliter of the extracting solution.

### Preparation Example 4: An Antibiotic Solution

2 mg of an antibiotic was weighed and placed into a 1.5 mL microcentrifuge tube, 1 mL of Tryptic Soy Broth (TSB) medium or Mueller Hinton II Broth (MH II Broth) medium was added into the microcentrifuge tube and mixed with the antibiotic inside a laminar flow cabinet to form a mixed solution, and the mixed solution was filtered using a filter with a pore size of 0.22 μm to obtain an antibiotic solution of Preparation Example 4. The antibiotic solution was prepared using florfenicol, doxycycline, cephalexin, amoxicillin, amoxicillin-clavulanic acid, oxytetracycline, or gentamicin, and the concentration of each antibiotic solution was approximately 2 mg/mL.

### Preparation Example 5: An Extract of Control Group

The method of Preparation Example 5 to obtain the extract is similar to Preparation Example 1, wherein the differences include: steps (a) and (b) were omitted, and the fermented coffee composition was not placed into the centrifuge tube in step (c); only 25 mL of 70% ethanol or water was added, shaken using the vortex mixer, and used in the subsequent steps. The extract of Preparation Example 5 was obtained essentially without any coffee composition but followed the same steps in extracting, serving as a sample for a control group.

### Example 1: An Anti-Bacterial Composition Comprising the Extract of Preparation Example 1 and an Antibiotic

The extract of Preparation Example 1 was mixed with one of the antibiotic solutions prepared from Preparation Example 4, and tryptic soy broth (TSB) medium was added for concentration adjustment to obtain an anti-bacterial composition of Example 1. The ratio of the coffee composition to the antibiotic in the anti-bacterial composition was in an amount of 1.5:1 to 25000:1.

### Example 2: An Anti-Bacterial Composition Comprising the Extract of Preparation Example 2 and an Antibiotic

The method of Example 2 to obtain the anti-bacterial composition is similar to Example 1, wherein the difference is the extract of Preparation Example 2 was mixed with one of the antibiotic solutions prepared from Preparation Example 4 to obtain an anti-bacterial composition of Example 2.

### Example 3: An Anti-Bacterial Composition Comprising the Extract of Preparation Example 3 and an Antibiotic

The method of Example 3 to obtain the anti-bacterial composition is similar to Example 1, wherein the difference is the extract of Preparation Example 3 was mixed with one of the antibiotic solutions prepared from Preparation Example 4 to obtain an anti-bacterial composition of Example 3.

The Examples and the Preparation Examples were used for testing the anti-bacterial effect.

### Test Example 1: Anti-Bacterial Synergistic Effect of the Anti-Bacterial Composition of Example 3 (Comprising the Ethanol Extract of the Unfermented Coffee Composition and the Antibiotic)

### 1. Colony Observation

1.5 g of TSB medium was weighed, placed into a 100 mL Erlenmeyer flask, and added with 50 mL of reverse osmosis (RO) water. The Erlenmeyer flask was then sterilized at 121° C. for 20 minutes and cooled to room temperature. Approximately 0.1 g of human Escherichia coli freeze-dried powder (purchased from the Food Industry Research and Development Institute, BCRC11509) was added into the Erlenmeyer flask, and the Erlenmeyer flask was placed in an incubator at a constant temperature of 37° C. and shaken at a speed of 120 rpm for 24 hours to obtain an Escherichia coli solution. According to Table 1 below, test groups 1-1 to 1-14 were prepared using the ethanol extract of Preparation Example 3 and antibiotics (florfenicol (FF) or doxycycline (DC)). 3 mL of the ethanol extract of Preparation Example 5 was used as a control group (C). 3 mL of the ethanol extract of Preparation Example 3 was used as the test groups 1-1 and 1-2. 3 mL of the antibiotic solution of Preparation Example 4 (an antibiotic solution of florfenicol or doxycycline, in which TSB medium was used for concentration adjustment) was used as the test groups 1-3, 1-6, 1-9, and 1-12. 3 mL of the anti-bacterial composition of Example 3 (concentrations and antibiotic types as shown in Table 1) was used as the test groups 1-4, 1-5, 1-7, 1-8, 1-10, 1-11, 1-13, and 1-14. N/A indicates “Not Applicable”.

### Table 1: Ethanol Extract Concentrations, Antibiotic Concentrations, and Antibiotic Types of Test Groups 1-1 to 1-14 (T1-1 to T1-14) and Control Group in

Test Example 1

| | Ethanol extract concentration | | |
|---|---|---|---|
| | of the unfermented coffee | | |
| | composition of Preparation | Antibiotic | Antibiotic |
| Groups | Example 3 | concentration | type |
| T1-1 | 6.25 mg/mL | 0 | N/A |
| T1-2 | 12.5 mg/mL | 0 | N/A |
| T1-3 | 0 | 10 μg/mL | FF |
| T1-4 | 6.25 mg/mL | 10 μg/mL | FF |
| T1-5 | 12.5 mg/mL | 10 μg/mL | FF |
| T1-6 | 0 | 20 μg/mL | FF |
| T1-7 | 6.25 mg/mL | 20 μg/mL | FF |
| T1-8 | 12.5 mg/mL | 20 μg/mL | FF |
| T1-9 | 0 | 10 μg/mL | DC |
| T1-10 | 6.25 mg/mL | 10 μg/mL | DC |
| T1-11 | 12.5 mg/mL | 10 μg/mL | DC |
| T1-12 | 0 | 20 μg/mL | DC |
| T1-13 | 6.25 mg/mL | 20 μg/ml | DC |
| T1-14 | 12.5 mg/mL | 20 μg/mL | DC |
| Control | Ethanol extract of Example 5 | 0 | N/A |
| group | | | |

The compositions of control group and test groups 1-1 to 1-14 were respectively placed into 15 screw cap tubes. The Escherichia coli solution was diluted with TSB medium, and 1 mL of the diluted Escherichia coli solution was added respectively to the control group and the test groups 1-1 to 1-14, making each group contain 1×10⁵ colony forming units (CFU)/mL of Escherichia coli. The mixture volume in each tube was 4 mL, and the tubes were placed in an incubator at a constant temperature of 37° C. and shaken at 120 rpm for 24 hours, forming Escherichia coli culture solutions of control group and test groups 1-1 to 1-14.

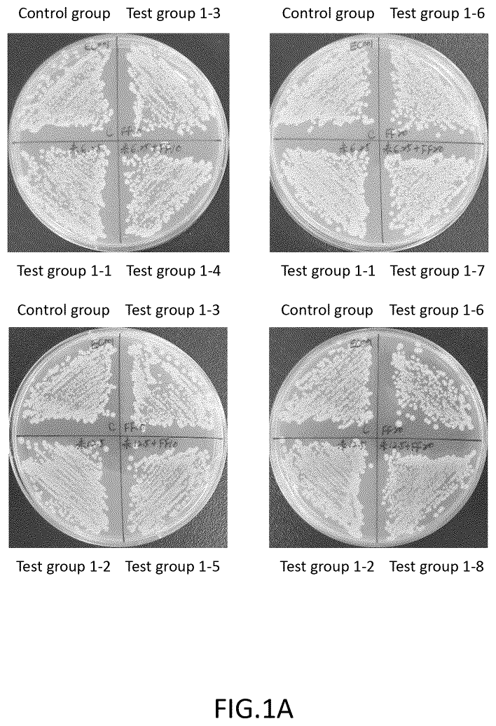

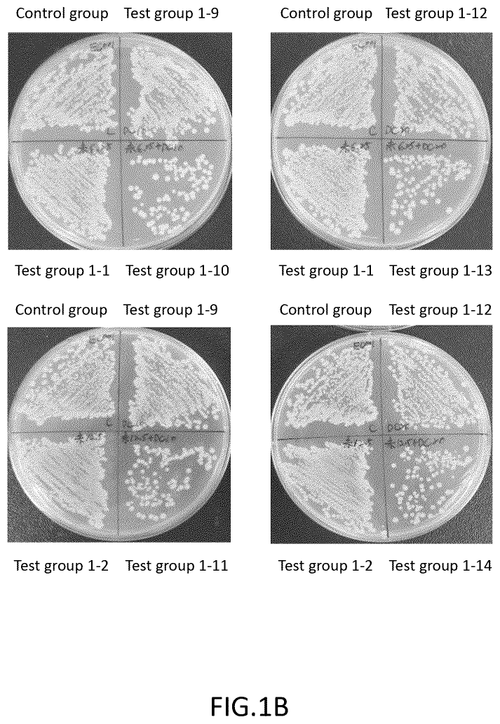

9 g of TSB medium and 4.5 g of agar were weighed, placed into a 500 mL serum bottle, and added with 300 mL of RO water. The serum bottle was then sterilized at 121° C. for 20 minutes to obtain a tryptic soy-agar (TSA) medium. After the TSA medium was cooled to 45° C. to 50° C., 20 mL of the TSA medium was poured into petri dishes, and four regions were marked under the bottom of each petri dish using a marker pen. The Escherichia coli culture solutions were 100-fold diluted with 0.85% saline solution to obtain diluted solutions of control group and test groups 1-1 to 1-14. 10 microliters (μL) of each diluted solution were inoculated to different regions of the petri dishes and uniformly spread using an inoculating loop. The inoculated petri dishes were inverted and cultured in an incubator at a constant temperature of 37° C. for 24 hours, and the colony distribution of Escherichia coli after culturing is shown in FIG. 1A and FIG. 1B.

### 2. Bacterial Number Calculation

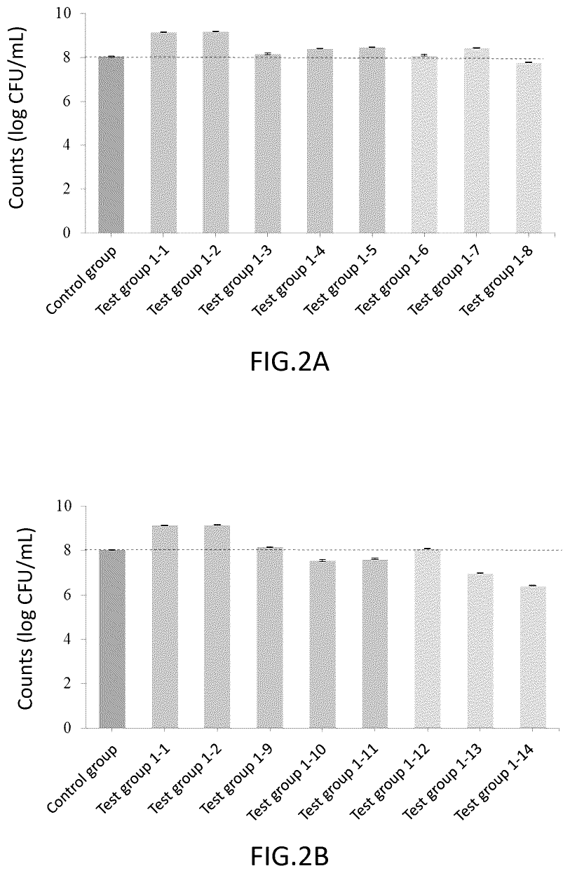

1 mL of the Escherichia coli culture solutions of each group were diluted using sequential 10-fold serial dilutions with 0.85% saline solution, and 1 mL of the diluted culture solutions at each gradient concentration were poured into the petri dishes. 20 mL of the TSA medium was added into each petri dish. After the liquid in the petri dishes was dried, the petri dishes were cultured at a constant temperature of 37° C. for 24 hours. The bacterial numbers in each petri dish were calculated based on the culture results. After multiplying back by the dilution factor, the calculated bacterial numbers of Escherichia coli in each group are shown in FIG. 2A and FIG. 2B.

In FIG. 1A and FIG. 2A, regardless of whether the ethanol extract of the unfermented coffee composition was comprised in the antibiotic solutions, the proliferation of Escherichia coli was not inhibited by the anti-bacterial compositions containing florfenicol (test groups 1-3 to 1-8). The anti-bacterial effect could not be achieved when the ethanol extract of the unfermented coffee composition and florfenicol were used simultaneously. In FIG. 1B and FIG. 2B, when the composition only contained the ethanol extract of Preparation Example (test groups 1-1, 1-2), the growth of Escherichia coli was not inhibited. However, in the anti-bacterial composition comprising doxycycline (test groups 1-9 to 1-14), the use of the anti-bacterial composition of test groups 1-10, 1-11, 1-13, and 1-14, which simultaneously comprised the ethanol extract of the unfermented coffee composition and doxycycline, resulted in the inhibition of Escherichia coli growth. This demonstrates that the ethanol extract of the unfermented coffee composition and doxycycline have an anti-bacterial synergistic effect and can achieve a better anti-bacterial effect when used simultaneously than doxycycline used alone (test groups 1-9, 1-12).

### Test Example 2: Anti-Bacterial Synergistic Effect of the Anti-Bacterial Composition of Example 1 (Comprising the Ethanol Extract of the Fermented Coffee Composition and the Antibiotic)

### 1. Colony Observation

1.5 g of TSB medium was weighed, placed into a 100 mL Erlenmeyer flask, and added with 50 mL of RO water. The Erlenmeyer flask was then sterilized at 121° C. for 20 minutes and cooled to room temperature. Approximately 0.1 g of human Escherichia coli freeze-dried powder (purchased from the Food Industry Research and Development Institute, BCRC11509) or human Salmonella enterica freeze-dried powder (purchased from the Food Industry Research and Development Institute, BCRC15464) was added into the Erlenmeyer flask, and the Erlenmeyer flask was placed in an incubator at a constant temperature of 37° C. and shaken at a speed of 120 rpm for 24 hours to obtain an Escherichia coli solution or a Salmonella enterica solution. According to Table 2 below, test groups 2-0 to 2-47 were prepared using the ethanol extract of Preparation Example 1 and antibiotics (cephalexin (CEX), amoxicillin (AMX), oxytetracycline (OTC), doxycycline (DC), florfenicol (FF), or gentamicin (GEN)). 3 mL of the ethanol extract of Preparation Example 5 was used as a control group (control or con). 3 mL of the ethanol extract of Preparation Example 1 (ME or M) was used as the test groups 2-0 and 2-1. In the other test groups, 3 mL of the antibiotic solution of Preparation Example 4 (an antibiotic solution of cephalexin, amoxicillin, oxytetracycline, doxycycline, florfenicol, or gentamicin, in which TSB medium was used for concentration adjustment) was used in the test groups with even numbers, and 3 mL of the anti-bacterial composition of Example 1 was used in the test groups with odd numbers (concentrations and antibiotic types as shown in Table 2). Herein, the fermented coffee composition of Preparation Example 1 used for the test groups 2-0 and 2-1 and other test groups with odd numbers was fermented for 5 days in the step (b).

### Table 2: Ethanol Extract Concentrations, Antibiotic Concentrations, and Antibiotic Types of Test Groups 2-0 to 2-47 (T2-0 to T2-47) and Control Group in

Test Example 2

| | Ethanol extract concentration | | | | |
|---|---|---|---|---|---|
| | of the fermented coffee | | | | |
| | composition of Preparation | | Antibiotic | | Antibiotic |
| Groups | Example 1 | | concentration | | type |
| T2-0 | 25 | mg/mL | 0 | | N/A |
| T2-1 | 12.5 | mg/mL | 0 | | N/A |
| T2-2 | 0 | | 250 | μg/mL | CEX |
| T2-3 | 12.5 | mg/mL | 250 | μg/mL | CEX |
| T2-4 | 0 | | 500 | μg/mL | CEX |
| T2-5 | 12.5 | mg/mL | 500 | μg/mL | CEX |
| T2-6 | 0 | | 1000 | μg/mL | CEX |
| T2-7 | 12.5 | mg/mL | 1000 | μg/mL | CEX |
| T2-8 | 0 | | 250 | μg/mL | AMX |
| T2-9 | 12.5 | mg/mL | 250 | μg/mL | AMX |
| T2-10 | 0 | | 500 | μg/mL | AMX |
| T2-11 | 12.5 | mg/mL | 500 | μg/mL | AMX |
| T2-12 | 0 | | 1000 | μg/mL | AMX |
| T2-13 | 12.5 | mg/mL | 1000 | μg/mL | AMX |
| T2-14 | 0 | | 2000 | μg/mL | AMX |
| T2-15 | 12.5 | mg/mL | 2000 | μg/mL | AMX |
| T2-16 | 0 | | 12.5 | μg/mL | OTC |
| T2-17 | 12.5 | mg/mL | 12.5 | μg/mL | OTC |
| T2-18 | 0 | | 25 | μg/mL | OTC |
| T2-19 | 12.5 | mg/mL | 25 | μg/mL | OTC |
| T2-20 | 0 | | 50 | μg/mL | OTC |
| T2-21 | 12.5 | mg/mL | 50 | μg/mL | OTC |
| T2-22 | 0 | | 100 | μg/mL | OTC |
| T2-23 | 12.5 | mg/mL | 100 | μg/mL | OTC |
| T2-24 | 0 | | 5 | μg/mL | DC |
| T2-25 | 12.5 | mg/mL | 5 | μg/mL | DC |
| T2-26 | 0 | | 10 | μg/mL | DC |
| T2-27 | 12.5 | mg/mL | 10 | μg/mL | DC |
| T2-28 | 0 | | 20 | μg/mL | DC |
| T2-29 | 12.5 | mg/mL | 20 | μg/mL | DC |
| T2-30 | 0 | | 50 | μg/mL | DC |
| T2-31 | 12.5 | mg/mL | 50 | μg/mL | DC |
| T2-32 | 0 | | 10 | μg/mL | FF |
| T2-33 | 25 | mg/mL | 10 | μg/mL | FF |
| T2-34 | 0 | | 20 | μg/mL | FF |
| T2-35 | 12.5 | mg/mL | 20 | μg/mL | FF |
| T2-36 | 0 | | 40 | μg/mL | FF |
| T2-37 | 12.5 | mg/mL | 40 | μg/mL | FF |
| T2-38 | 0 | | 80 | μg/mL | FF |
| T2-39 | 12.5 | mg/mL | 80 | μg/mL | FF |
| T2-40 | 0 | | 1 | μg/mL | GEN |
| T2-41 | 12.5 | mg/mL | 1 | μg/mL | GEN |
| T2-42 | 0 | | 2 | μg/mL | GEN |
| T2-43 | 12.5 | mg/mL | 2 | μg/mL | GEN |
| T2-44 | 0 | | 4 | μg/mL | GEN |
| T2-45 | 12.5 | mg/mL | 4 | μg/mL | GEN |
| T2-46 | 0 | | 8 | μg/mL | GEN |
| T2-47 | 12.5 | mg/mL | 8 | μg/mL | GEN |
| Control | Ethanol extract of Example 5 | | 0 | | N/A |
| group | | | | | |

The compositions of control group and test groups 2-0 to 2-47 were respectively placed into 49 screw cap tubes. The Escherichia coli solution or the Salmonella enterica solution was diluted with TSB medium (diluted 10-fold sequentially with a sterile saline solution to obtain a 10²-fold diluted solution, and 10 μl of the 10²-fold diluted solution was inoculated with a final dilution factor of 10⁴), and 1 mL of the diluted Escherichia co/i solution or the diluted Salmonella enterica solution was added respectively to the control group and the test groups 2-0 to 2-47, making each group contain 1×10⁵ CFU/mL of Escherichia co/i or Salmonella enterica. The mixture volume in each tube was 4 mL, and the tubes were placed in an incubator at a constant temperature of 37° C. and shaken at 120 rpm for 24 hours, forming Escherichia coli culture solutions or Salmonella enterica culture solutions of control group and test groups 2-0 to 2-47.

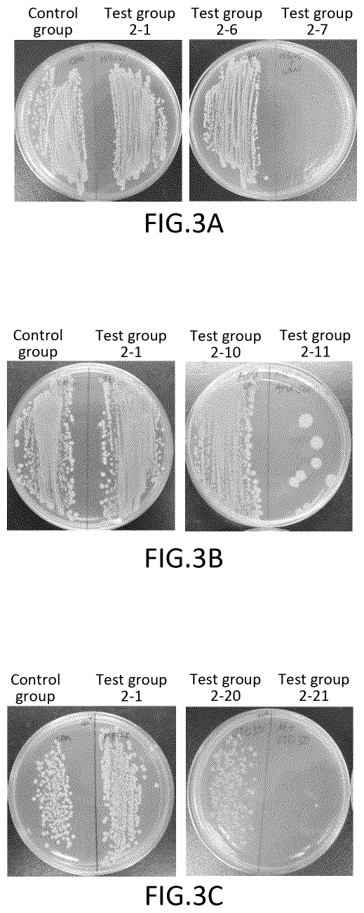

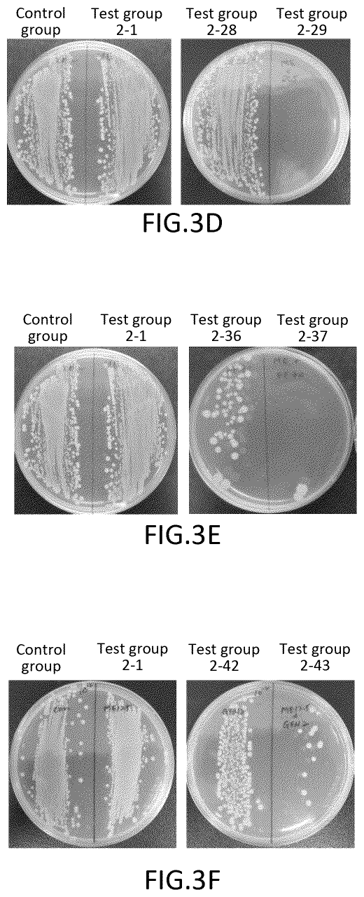

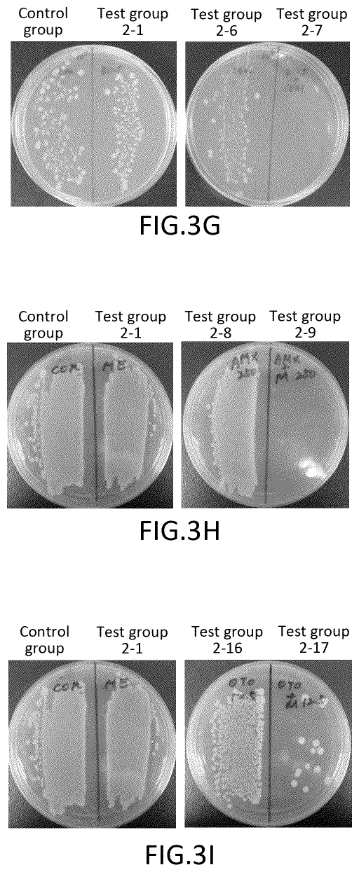

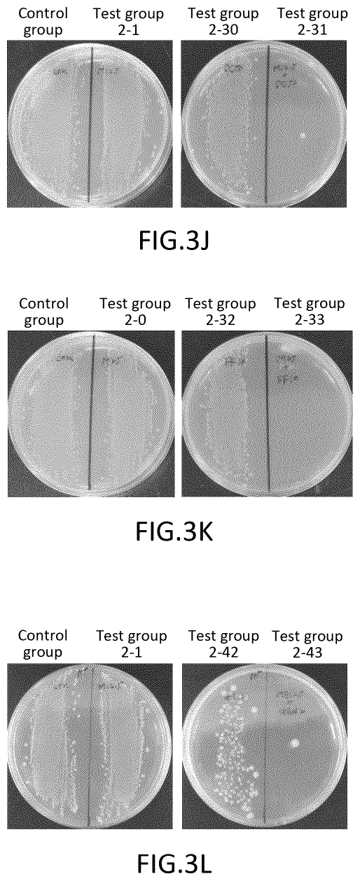

15 g of TSB medium and 7.5 g of agar were weighed, placed into a 1000 mL serum bottle, and added with 500 mL of RO water. The serum bottle was then sterilized at 121° C. for 20 minutes to obtain a TSA medium. After the TSA medium was cooled to 45° C. to 50° C., 20 mL of the TSA medium was poured into petri dishes, and two regions were marked under the bottom of each petri dish using a marker pen. The Escherichia coli culture solutions or the Salmonella enterica culture solutions were 100-fold diluted with 0.85% saline solution to obtain diluted solutions of control group and test groups 2-0 to 2-47 comprising Escherichia coli or Salmonella enterica. 10 μL of the diluted solutions of control group and test groups 2-1, 2-6, 2-7, 2-10, 2-11, 2-20, 2-21, 2-28, 2-29, 2-36, 2-37, 2-42, and 2-43 comprising Escherichia coli, and the diluted solutions of control group and test groups 2-0, 2-1, 2-6 to 2-9, 2-16, 2-17, 2-30 to 2-33, 2-42, and 2-43 comprising Salmonella enterica, were inoculated to different regions of the petri dishes and uniformly spread using an inoculating loop. The inoculated petri dishes were inverted and cultured in an incubator at a constant temperature of 37° C. for 24 hours. The colony distribution of Escherichia coli after culturing is shown in FIG. 3A to FIG. 3F, and the colony distribution of Salmonella enterica after culturing is shown in FIG. 3G to FIG. 3L.

### 2. Bacterial Number Calculation

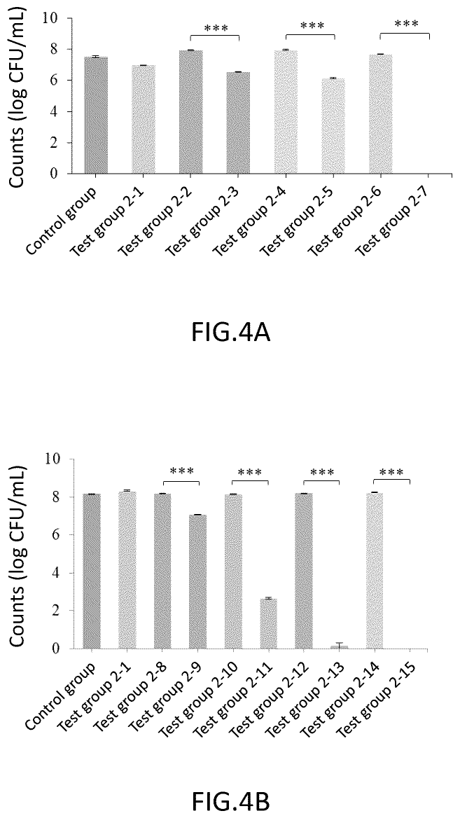

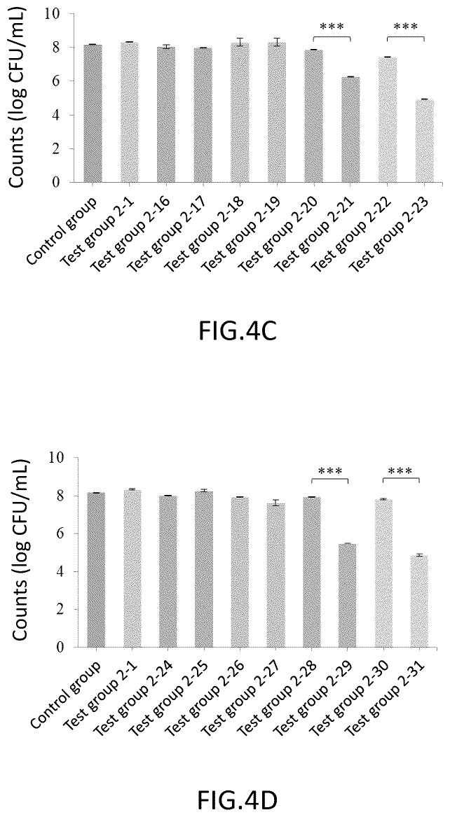

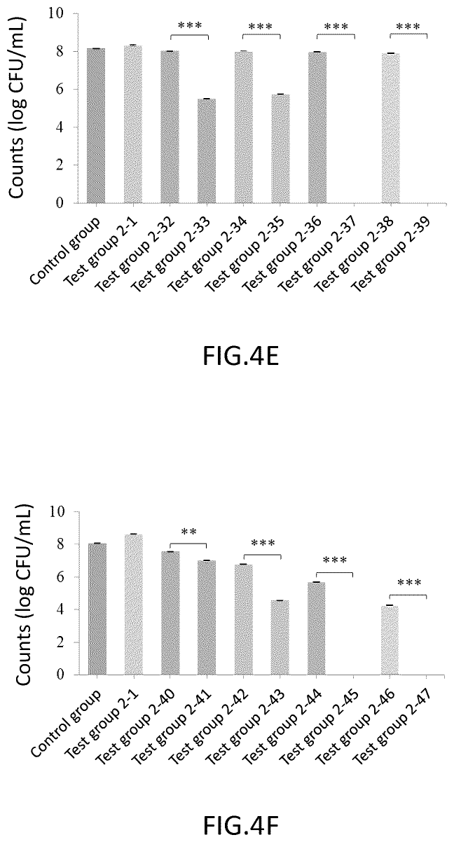

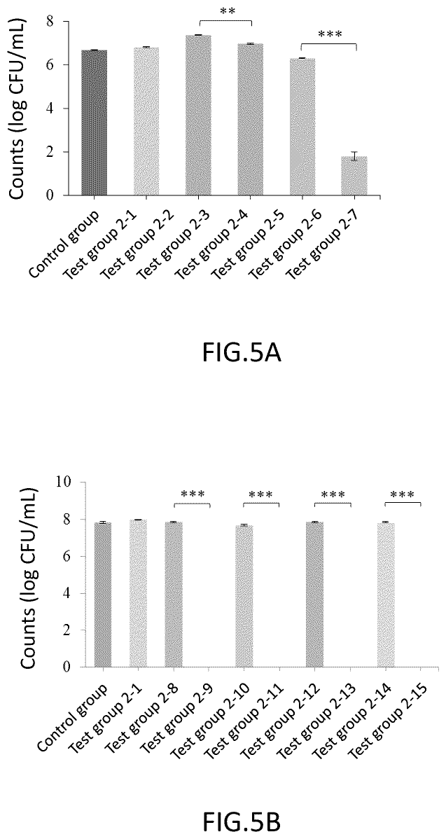

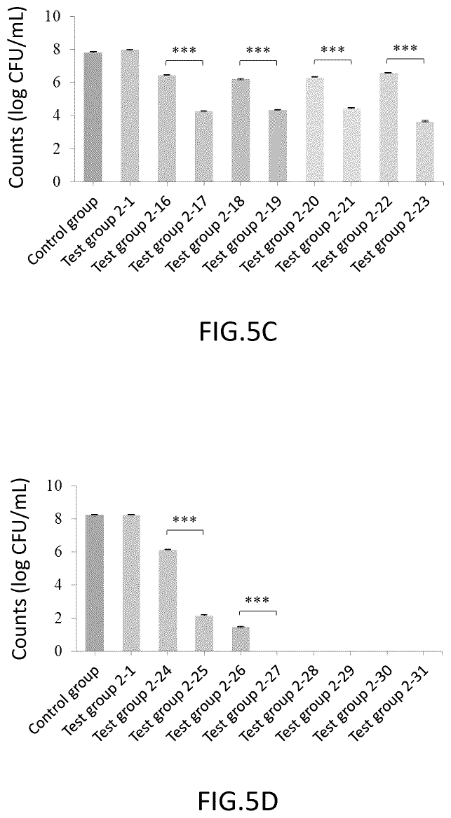

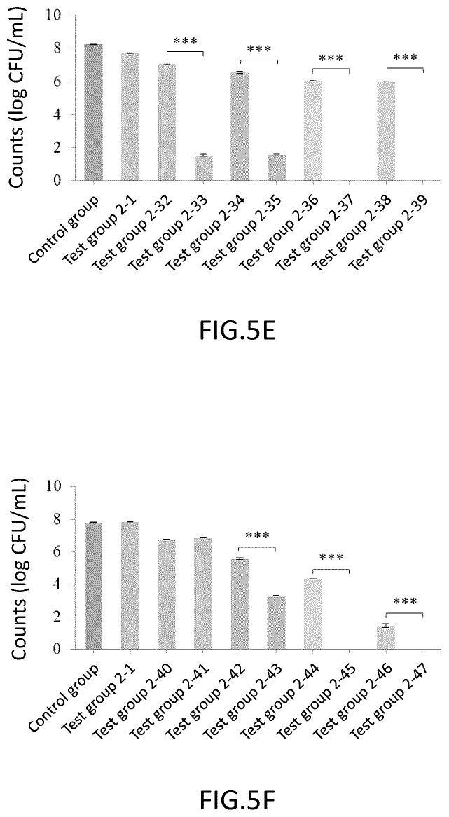

1 mL of the Escherichia coli culture solutions or the Salmonella enterica culture solutions of each group were diluted using sequential 10-fold serial dilutions with 0.85% saline solution, and 1 mL of the diluted culture solutions at each gradient concentration were poured into the petri dishes. 20 mL of the TSA medium was added into each petri dish. After the liquid in the petri dishes was dried, the petri dishes were cultured at a constant temperature of 37° C. for 24 hours. The bacterial numbers in each petri dish were calculated based on the culture results. After multiplying back by the dilution factor, the calculated bacterial numbers of Escherichia coli in each group are shown in FIG. 4A to FIG. 4F, and the calculated bacterial numbers of Salmonella enterica in each group are shown in FIG. 5A to FIG. 5F, where * indicates P<0.05, ** indicates P<0.01, and *** indicates P<0.001.

Besides, bacterial numbers and log reductions relative to the control group in specific test groups were calculated using two replicates for each group. The results of Escherichia coli are shown in Table 3 below, and the results of Salmonella enterica are shown in Table 4 below.

TABLE 3
Bacterial numbers and log reductions of Escherichia coli

| | | | | Log reduction |
|---|---|---|---|---|
| | Antibiotic | | Bacterial number | (relative to the |
| Groups | concentration and type | | (log CFU/mL) | control group) |
| Control | N/A | | 8.20 ± 0.02 | N/A |
| group | | | | |
| T2-1 | N/A | | 8.33 ± 0.03 | −0.13 |
| T2-4 | CEX, 500 | μg/mL | 7.97 ± 0.02 | 0.23 |
| T2-5 | CEX, 500 | μg/mL | 6.15 ± 0.01 | 2.05 |
| T2-10 | AMX, 500 | μg/mL | 8.16 ± 0.02 | 0.04 |
| T2-11 | AMX, 500 | μg/mL | 2.65 ± 0.05 | 5.55 |
| T2-20 | OTC, 50 | μg/mL | 7.88 ± 0.02 | 0.32 |
| T2-21 | OTC, 50 | μg/mL | 6.26 ± 0.01 | 1.94 |
| T2-28 | DC, 20 | μg/mL | 7.94 ± 0.02 | 0.26 |
| T2-29 | DC, 20 | μg/mL | 5.48 ± 0.00 | 2.72 |
| T2-36 | FF, 40 | μg/mL | 7.99 ± 0.01 | 0.21 |
| T2-37 | FF, 40 | μg/mL | 0.00 ± 0.00 | 8.20 |
| T2-44 | GEN, 4 | μg/mL | 5.69 ± 0.00 | 2.51 |
| T2-45 | GEN, 4 | μg/mL | 0.00 ± 0.00 | 8.20 |

TABLE 4
Bacterial numbers and log reductions of Salmonella enterica

| | | | | Log reduction |
|---|---|---|---|---|
| | Antibiotic | | Bacterial number | (relative to the |
| Groups | concentration and type | | (log CFU/mL) | control group) |
| Control | N/A | | 7.83 ± 0.04 | N/A |
| group | | | | |
| T2-1 | N/A | | 8.00 ± 0.00 | −0.17 |
| T2-6 | CEX, 1000 | μg/mL | 8.13 ± 0.04 | −0.30 |
| T2-7 | CEX, 1000 | μg/mL | 2.95 ± 0.20 | 4.88 |
| T2-8 | AMX, 250 | μg/mL | 7.85 ± 0.02 | −0.02 |
| T2-9 | AMX, 250 | μg/mL | 0.00 ± 0.00 | 7.83 |
| T2-16 | OTC, 12.5 | μg/mL | 6.48 ± 0.02 | 1.35 |
| T2-17 | OTC, 12.5 | μg/mL | 4.28 ± 0.01 | 3.55 |
| T2-30 | DC, 50 | μg/mL | 6.15 ± 0.05 | 2.10 |
| T2-31 | DC, 50 | μg/mL | 2.19 ± 0.04 | 6.06 |
| T2-32 | FF, 10 | μg/mL | 7.05 ± 0.01 | 1.20 |
| T2-33 | FF, 10 | μg/mL | 1.54 ± 0.06 | 6.71 |
| T2-44 | GEN, 4 | μg/mL | 4.34 ± 0.00 | 3.49 |
| T2-45 | GEN, 4 | μg/mL | 0.00 ± 0.00 | 7.83 |

In FIG. 3A to FIG. 3L and Tables 3 to 4, when the anti-bacterial compositions comprised the ethanol extract of the fermented coffee composition prepared from the raw coffee beans or the roasted coffee beans, the anti-bacterial compositions with any one of the six antibiotics used in this Test Example may exhibit anti-bacterial synergistic effects. The colony distributions of Escherichia coli (test groups 2-7, 2-11, 2-21, 2-29, 2-37, and 2-43) and Salmonella enterica (test groups 2-7, 2-9, 2-17, 2-31, 2-33, and 2-43) after culturing were sparse, and the bacterial numbers of Escherichia coli (test groups 2-5, 2-11, 2-21, 2-29, 2-37, and 2-45) and Salmonella enterica (test groups 2-7, 2-9, 2-17, 2-31, 2-33, and 2-45) could be reduced, showing that the anti-bacterial compositions simultaneously comprising the ethanol extract of the present invention and the antibiotics have an anti-bacterial effect against Escherichia coli and Salmonella enterica. The anti-bacterial synergistic effect formed by the combination of the ethanol extract and the antibiotics can also be seen in FIG. 4A to FIG. 4F and FIG. 5A to FIG. 5F.

### Test Example 3: Anti-Bacterial Synergistic Effect of the Anti-Bacterial Composition of Example 2 (Comprising the Ethanol Extract of the Fermented Coffee Composition and the Antibiotic Amoxicillin)

The method of Test Example 3 is approximately the same as the colony observation in Test Example 1, wherein the differences include: in Test Example 3, according to Table 5 below, test groups 3-1 to 3-7 were prepared using the ethanol extract of Preparation Example 2 (with different fermenting times) and amoxicillin (AMX) and respectively placed into 7 screw cap tubes. Besides, 3 g of TSB medium and 1.5 g of agar were weighed, placed into a 500 mL serum bottle, added with 100 mL of RO water, and then sterilized to obtain the TSA medium. 3 mL of the ethanol extract of Preparation Example 5 was used as a control group (C). 3 mL of the antibiotic solution of Preparation Example 4 (an antibiotic solution of amoxicillin, in which TSB medium was used for concentration adjustment to reach 1000 μg/mL in the antibiotic solution) was used as the test group 3-1. 3 mL of the ethanol extract of Preparation Example 2 was used as the test groups 3-2, 3-4, and 3-6. 3 mL of the anti-bacterial composition of Example 2 was used as the test groups 3-3, 3-5, and 3-7. Herein, the fermented coffee composition of Preparation Example 2 used for the test groups 3-2 to 3-7 was obtained from different fermenting times in the step (b) (with fermenting times as shown in Table 5).

TABLE 5
Ethanol extract concentrations, coffee composition fermenting times,
antibiotic concentrations, and antibiotic types of test groups
3-1 to 3-7 (T3-1 to T3-7) and control group in Test Example 3
(F2: the fermented coffee composition of Preparation Example 2)

| | Ethanol extract | Fermenting | Antibiotic | Antibiotic |
|---|---|---|---|---|
| Groups | concentration of F2 | time of F2 | concentration | type |
| T3-1 | 0 | N/A | 1000 μg/mL | AMX |
| T3-2 | 3.125 mg/mL | 3 days | 0 | N/A |
| T3-3 | 3.125 mg/mL | 3 days | 1000 μg/mL | AMX |
| T3-4 | 3.125 mg/mL | 4 days | 0 | N/A |
| T3-5 | 3.125 mg/mL | 4 days | 1000 μg/mL | AMX |
| T3-6 | 3.125 mg/mL | 5 days | 0 | N/A |
| T3-7 | 3.125 mg/mL | 5 days | 1000 μg/mL | AMX |
| Control | Ethanol extract of | N/A | 0 | N/A |
| group | Example 5 | | | |

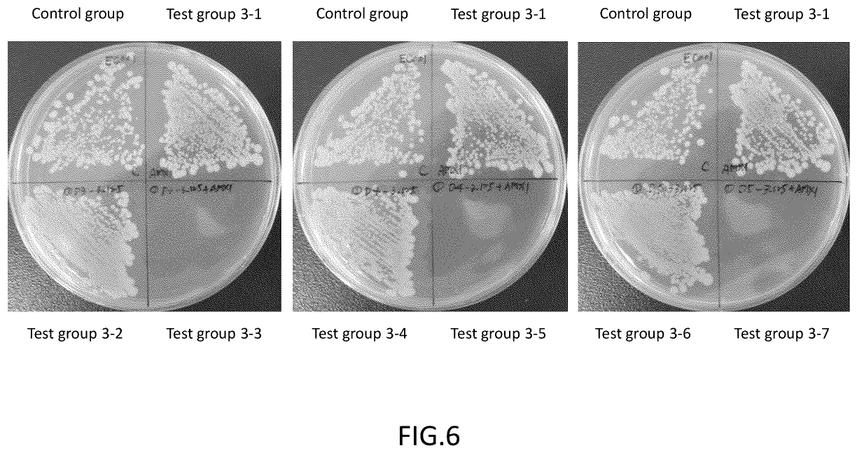

The colony distribution of Escherichia co/i after culturing is shown in FIG. 6. When the anti-bacterial compositions comprised the ethanol extract of the fermented coffee composition prepared from the recycled coffee grounds (test groups 3-3, 3-5, and 3-7), an anti-bacterial synergistic effect of the ethanol extract and amoxicillin could effectively inhibit the growth of Escherichia coli whether the fermented coffee composition were fermented for 3 days, 4 days, or 5 days, achieving a better anti-bacterial effect compared to the ethanol extract alone (test groups 3-2, 3-4, and 3-6).

### Test Example 4: Anti-Bacterial Synergistic Effect of the Anti-Bacterial Composition of Example 1 (Comprising the Water Extract of the Fermented Coffee Composition and the Antibiotic)

The method of Test Example 4 is approximately the same as Test Example 1, wherein the differences include: in Test Example 4, according to Table 6 below, test groups 4-1 to 4-15 were prepared using the water extract of Preparation Example 1 (the coffee beans are different types of whole beans, grits, or powder) and antibiotics (cephalexin (CEX), florfenicol (FF), or doxycycline (DC)) and respectively placed into 15 screw cap tubes. Besides, 6 g of TSB medium and 3 g of agar were weighed, placed into a 500 mL serum bottle, added with 200 mL of RO water, and then sterilized to obtain the TSA medium. 3 mL of the water extract of Preparation Example 5 was used as a control group (con). 3 mL of the water extract of Preparation Example 1 was used as the test groups 4-1 to 4-3. 3 mL of the antibiotic solution of Preparation Example 4 (an antibiotic solution of cephalexin, florfenicol, or doxycycline, in which TSB medium was used for concentration adjustment) was used as the test groups 4-4, 4-8, and 4-12. 3 mL of the anti-bacterial composition of Example 1 was used as the test groups 4-5 to 4-7, 4-9 to 4-11, and 4-13 to 4-15 (concentrations and antibiotic types as shown in Table 6). Herein, when the water extract of Preparation Example 1 and the anti-bacterial composition of Example 1 were prepared, the fermented coffee composition of Preparation Example 1 used for the test groups 4-1 to 4-3, 4-5 to 4-7, 4-9 to 4-11, and 4-13 to 4-15 was obtained using different types of the raw coffee beans in the step (a) (with coffee bean types as shown in Table 6) and fermented for 5 days in the step (b).

TABLE 6
Water extract concentrations, raw coffee bean types of
coffee composition, antibiotic concentrations, and antibiotic
types of test groups 4-1 to 4-15 (T4-1 to T4-15) and
control group in Test Example 4 (F1: the fermented coffee
composition of Preparation Example 1)

| | Water | | | | |
|---|---|---|---|---|---|
| | extract | Raw coffee | | | |
| | concentration | bean type | Antibiotic | | Antibiotic |
| Groups | of F1 | of F1 | concentration | | type |
| T4-1 | 12.5 mg/mL | Whole coffee | 0 | | N/A |
| | | beans | | | |
| T4-2 | 12.5 mg/mL | Coffee bean | 0 | | N/A |
| | | grits | | | |
| T4-3 | 12.5 mg/mL | Coffee powder | 0 | | N/A |
| T4-4 | 0 | N/A | 1000 | μg/mL | CEX |
| T4-5 | 12.5 mg/mL | Whole coffee | 1000 | μg/mL | CEX |
| | | beans | | | |
| T4-6 | 12.5 mg/mL | Coffee bean | 1000 | μg/mL | CEX |
| | | grits | | | |
| T4-7 | 12.5 mg/mL | Coffee powder | 1000 | μg/mL | CEX |
| T4-8 | 0 | N/A | 20 | μg/mL | FF |
| T4-9 | 12.5 mg/mL | Whole coffee | 20 | μg/mL | FF |
| | | beans | | | |
| T4-10 | 12.5 mg/mL | Coffee bean | 20 | μg/mL | FF |
| | | grits | | | |
| T4-11 | 12.5 mg/mL | Coffee powder | 20 | μg/mL | FF |
| T4-12 | 0 | N/A | 20 | μg/mL | DC |
| T4-13 | 12.5 mg/mL | Whole coffee | 20 | μg/mL | DC |
| | | beans | | | |
| T4-14 | 12.5 mg/mL | Coffee bean | 20 | μg/mL | DC |
| | | grits | | | |
| T4-15 | 12.5 mg/mL | Coffee powder | 20 | μg/mL | DC |
| Control | Water | N/A | 0 | | N/A |
| group | extract of | | | | |
| | Example 5 | | | | |

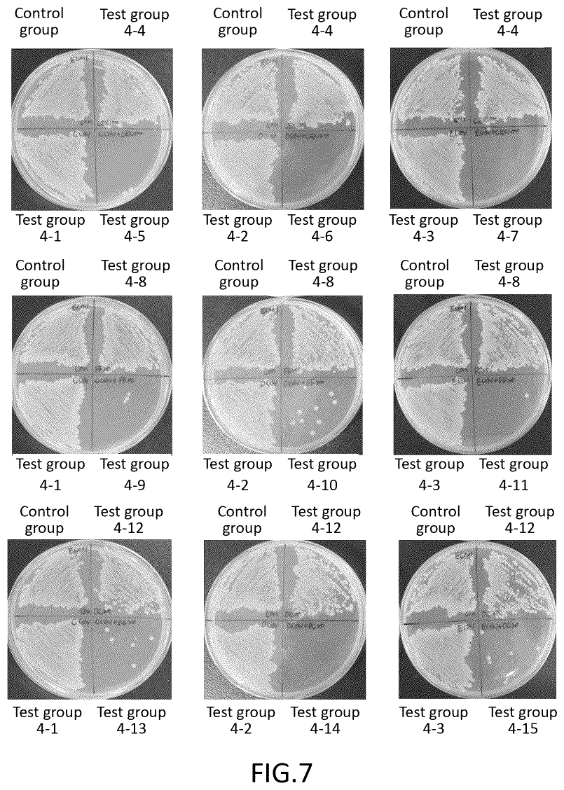

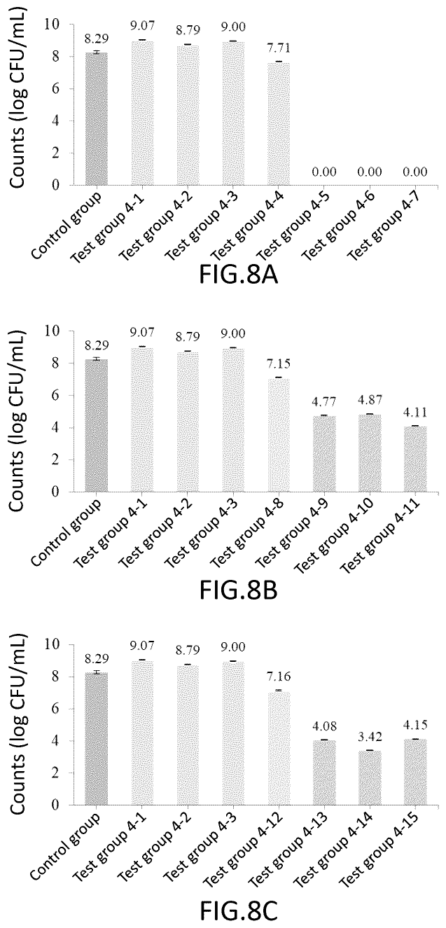

The colony distribution of Escherichia coli after culturing is shown in FIG. 7, and the calculated bacterial numbers of Escherichia coli in each test group are shown in FIG. 8A to FIG. 8C. In FIG. 7 and FIG. 8A to FIG. 8C, when the anti-bacterial compositions comprised the water extract of the fermented coffee composition prepared from the raw coffee beans (test groups 4-5 to 4-7, 4-9 to 4-11, and 4-13 to 4-15), an anti-bacterial synergistic effect of the water extract and cephalexin, florfenicol, and doxycycline could effectively inhibit the growth of Escherichia coli whether the raw coffee beans were whole coffee beans (C), coffee bean grits (D), or coffee powder (E), achieving a better anti-bacterial effect compared to the antibiotic alone (test groups 4-4, 4-8, and 4-12).

### Test Example 5: Minimum Inhibitory Concentration of the Anti-Bacterial Composition of Example 1 (Comprising the Ethanol Extract of the Fermented Coffee Composition and the Antibiotic) Against Drug-Resistant Bacteria

A Mueller Hinton II broth (MH II Broth) with a concentration of 22 g/liter (L) was prepared, sterilized at 121° C. for 15 minutes, and cooled to room temperature. The sterilized MH II Broth was added to the second columns to the twelfth columns of 96-well plates. According to Table 7 below, test groups 5-1 to 5-9 were prepared using the ethanol extract of Preparation Example 1 and antibiotics (florfenicol (FF), enrofloxacin (EF), or doxycycline (DC)). The antibiotic solution of Preparation Example 4 (an antibiotic solution of florfenicol, enrofloxacin, or doxycycline, in which MH II Broth was used for concentration adjustment) was used as the test groups 5-1, 5-4, and 5-7. The anti-bacterial composition of Example 1 (concentrations and antibiotic types as shown in Table 7) was used as the test groups 5-2, 5-3, 5-5, 5-6, 5-8, and 5-9. Herein, the fermented coffee composition of Preparation Example 1 used for the test groups 5-2, 5-3, 5-5, 5-6, 5-8, and 5-9 was fermented for 5 days in the step (b).

TABLE 7
Ethanol extract concentrations, antibiotic concentrations, and antibiotic
types of test groups 5-1 to 5-9 (T5-1 to T5-9) in Test Example 5

| | Ethanol extract concentration | | | | |
|---|---|---|---|---|---|
| | of the fermented coffee | | | | |
| | composition of Preparation | | Antibiotic | | Antibiotic |
| Groups | Example 1 | | concentration | | type |
| T5-1 | 0 | | 1024 | μg/mL | FF |
| T5-2 | 12.5 | mg/mL | 1024 | μg/mL | FF |
| T5-3 | 25 | mg/mL | 1024 | μg/mL | FF |
| T5-4 | 0 | | 512 | μg/mL | EF |
| T5-5 | 12.5 | mg/mL | 512 | μg/mL | EF |
| T5-6 | 25 | mg/mL | 512 | μg/mL | EF |
| T5-7 | 0 | | 512 | μg/mL | DC |
| T5-8 | 12.5 | mg/mL | 512 | μg/mL | DC |
| T5-9 | 25 | mg/mL | 512 | μg/mL | DC |

200 μL of the anti-bacterial compositions or the antibiotic solutions of test groups 5-1 to 5-9 were respectively added to the first columns (column 1) of the 96-well plates. 100 μL of the solutions in each well were transferred from the column “n” to the column “n+1” until the solutions were added to the test groups 5-1 to 5-9 in the twelfth columns, wherein n was an integer starting from 1 to 11. The concentration of florfenicol of the test groups 5-1 to 5-3 in the twelfth columns was 0.5 μg/mL, the concentration of enrofloxacin of the test groups 5-4 to 5-6 in the twelfth columns was 0.25 μg/mL, and the concentration of doxycycline of the test groups 5-7 to 5-9 in the twelfth columns was 0.25 μg/mL. Based on the 0.5 McFarland Standard, drug-resistant porcine Escherichia coli, chicken Escherichia coli, porcine Salmonella enterica, chicken Salmonella enterica, Pasteurella multocida, and Glaesserella parasuis (provided by the Department of Veterinary Medicine at National Chiayi University) were used to prepare bacterial solutions with a concentration of 1.5×10⁸ CFU/mL. 5 μL of the bacterial solutions were respectively added to each well of the 96-well plates. The 96-well plates were cultured in an incubator at a constant temperature of 37° C. for 16 hours to 24 hours, and minimum inhibitory concentrations (MIC) of each test group were then determined.

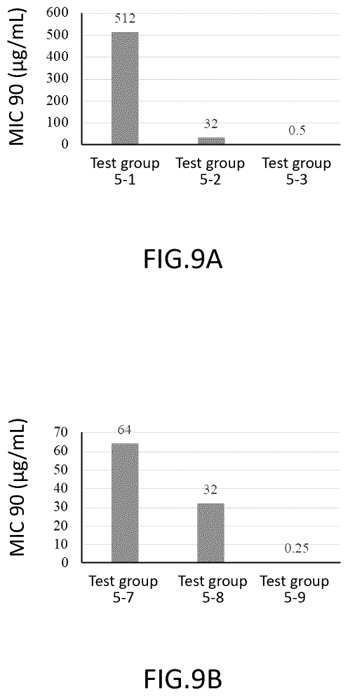

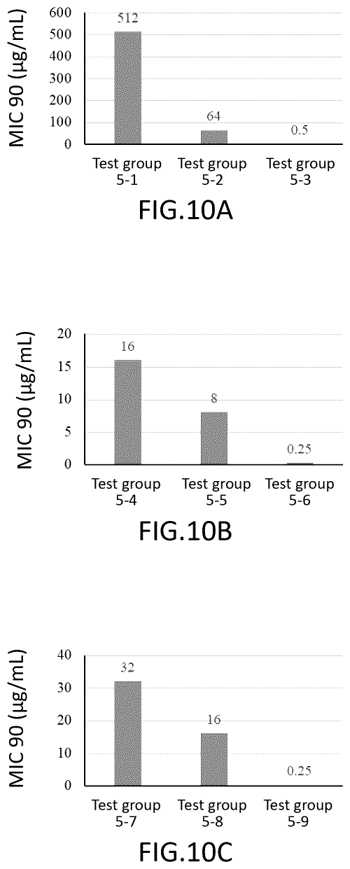

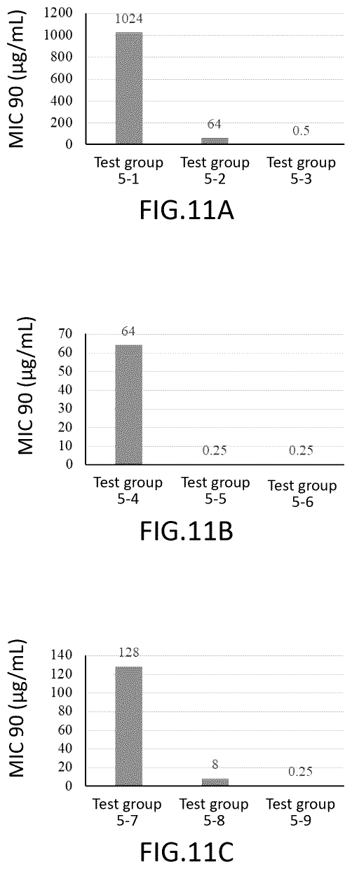

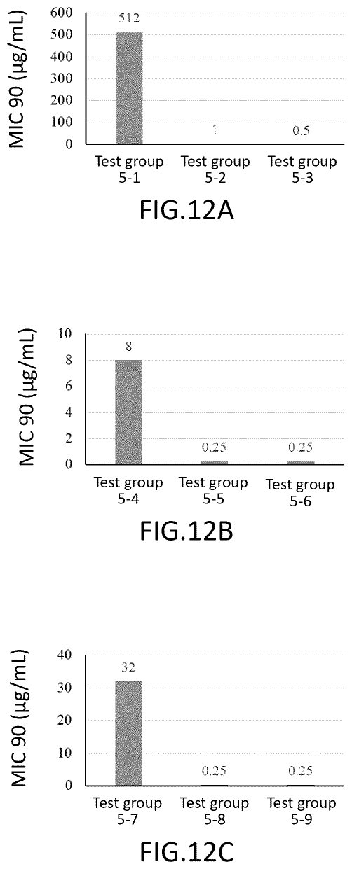

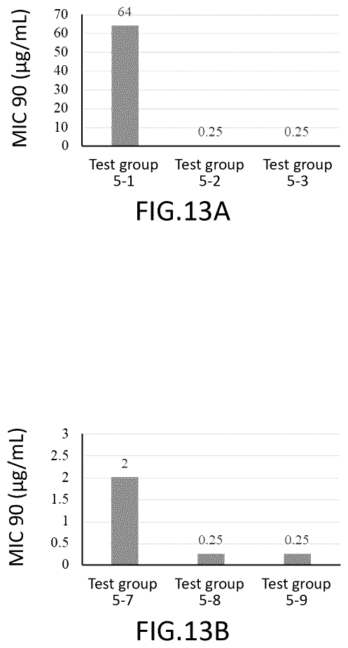

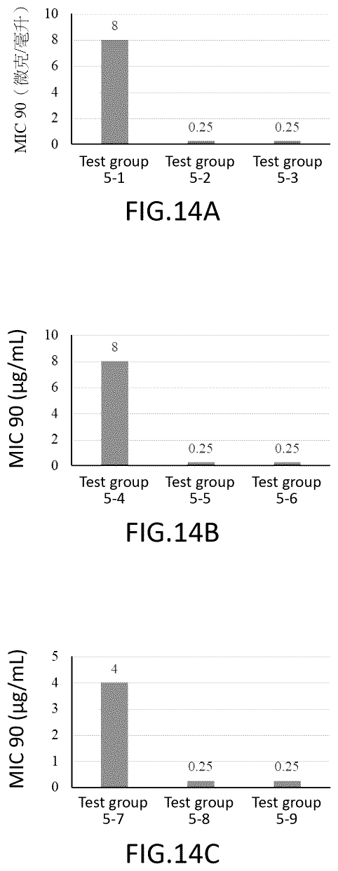

The concentrations of the anti-bacterial compositions or the antibiotic solutions of test groups 5-1 to 5-3 and 5-7 to 5-9 to achieve an inhibition effect against 90% bacterial growth (MIC 90) of the drug-resistant porcine Escherichia coli are shown in FIG. 9A to FIG. 9B. The concentrations of the anti-bacterial compositions or the antibiotic solutions of test groups 5-1 to 5-9 to achieve MIC 90 of the drug-resistant chicken Escherichia coli are shown in FIG. 10A to FIG. 10C. The concentrations of the anti-bacterial compositions or the antibiotic solutions of test groups 5-1 to 5-9 to achieve MIC 90 of the drug-resistant porcine Salmonella enterica are shown in FIG. 11A to FIG. 11C. The concentrations of the anti-bacterial compositions or the antibiotic solutions of test groups 5-1 to 5-9 to achieve MIC 90 of the drug-resistant chicken Salmonella enterica are shown in FIG. 12A to FIG. 12C. The concentrations of the anti-bacterial compositions or the antibiotic solutions of test groups 5-1 to 5-3 and 5-7 to 5-9 to achieve MIC 90 of the drug-resistant Pasteurella multocida are shown in FIG. 13A to FIG. 13B. The concentrations of the anti-bacterial compositions or the antibiotic solutions of test groups 5-1 to 5-9 to achieve MIC 90 of the drug-resistant Glaesserella parasuis are shown in FIG. 14A to FIG. 14C.

According to the results in FIG. 9A to FIG. 14C, when florfenicol, enrofloxacin, or doxycycline was used alone (test groups 5-1, 5-4, and 5-7), the concentration of the antibiotic to achieve an inhibition effect against 90% bacterial number was higher. When a combination of each antibiotic and the fermented coffee composition was used (test groups 5-2, 5-3, 5-5, 5-6, 5-8, and 5-9), the concentration of each antibiotic to achieve MIC 90 could be significantly reduced. Moreover, the concentrations to achieve MIC 90 of the drug-resistant porcine Escherichia coli, chicken Escherichia coli, and porcine Salmonella enterica exhibit a more pronounced decreasing trend as the concentration of the ethanol extract of the fermented coffee composition increases. Therefore, the weakened anti-bacterial effect of antibiotics due to drug resistance could be enhanced through the synergistic effect with the ethanol extract of the fermented coffee composition.

### Test Example 6: Anti-Bacterial Effect of the Ethanol Extract of the Fermented Coffee Composition and the Antibiotic Against Various Bacteria Types

1.5 g of TSB medium or 2.75 g of lactobacilli MRS broth (MRSB) was weighed, placed into a 100 mL Erlenmeyer flask, and added with 50 mL of RO water. The Erlenmeyer flask was then sterilized at 121° C. for 20 minutes and cooled to room temperature. Approximately 0.1 g of human Escherichia coli freeze-dried powder (purchased from the Food Industry Research and Development Institute, BCRC11509) or human Salmonella enterica freeze-dried powder (purchased from the Food Industry Research and Development Institute, BCRC15464) was added into the Erlenmeyer flask containing TSB medium; approximately 0.1 g of porcine Lactobacillus pentosus freeze-dried powder (a strain selected by King's Ground Biotech, BL010, identified by Tri-I Biotech) or porcine Lactobacillus reuteri freeze-dried powder (a strain selected by King's Ground Biotech, BL011, identified by Tri-I Biotech) was added into the Erlenmeyer flask containing MRSB. The Erlenmeyer flask was placed in an incubator at a constant temperature of 37° C. The Erlenmeyer flask containing Escherichia coli or Salmonella enterica was shaken at a speed of 120 rpm for 24 hours to obtain an Escherichia coli solution or a Salmonella enterica solution. The Erlenmeyer flask containing Lactobacillus pentosus or Lactobacillus reuteri was cultured for 24 hours to obtain a Lactobacillus pentosus solution or a Lactobacillus reuteri solution. According to Table 8 below, test groups 6-1 to 6-6 were prepared using the ethanol extract of Preparation Example 1 and oxytetracycline (OTC). 3 mL of the ethanol extract of Preparation Example 5 was used as a control group. 3 mL of the ethanol extract of Preparation Example 1 was used as the test groups 6-1 to 6-3. 3 mL of the antibiotic solution of Preparation Example 4 (an antibiotic solution of oxytetracycline, in which TSB medium or MRSB was used for concentration adjustment) was used as the test groups 6-4 to 6-6. Herein, the fermented coffee composition of Preparation Example 1 used for the test groups 6-1 to 6-3 was fermented for 5 days in the step (b).

TABLE 8
Ethanol extract concentrations, antibiotic concentrations,
and antibiotic types of test groups 6-1 to 6-6 (T6-1
to T6-6) and control group in Test Example 6

| | Ethanol extract concentration | | | |
|---|---|---|---|---|
| | of the fermented coffee | | | |
| | composition of Preparation | | Antibiotic | Antibiotic |
| Groups | Example 1 | | concentration | type |
| T6-1 | 12.5 | mg/mL | 0 | N/A |
| T6-2 | 25 | mg/mL | 0 | N/A |
| T6-3 | 50 | mg/mL | 0 | N/A |
| T6-4 | 0 | | 0.1 μg/mL | OTC |
| T6-5 | 0 | | 0.2 μg/mL | OTC |
| T6-6 | 0 | | 0.3 μg/mL | OTC |
| Control | Ethanol extract of Example 5 | | 0 | N/A |
| group | | | | |

The ethanol extracts or the antibiotic solutions of control group and test groups 6-1 to 6-6 were respectively placed into 7 screw cap tubes. The Escherichia co/i solution or the Salmonella enterica solution was diluted with TSB medium. The Lactobacillus pentosus solution or the Lactobacillus reuteri solution was diluted with MRSB. 1 mL of the diluted Escherichia co/i solution, the diluted Salmonella enterica solution, the Lactobacillus pentosus solution, or the Lactobacillus reuteri solution was added respectively to the control group and the test groups 6-1 to 6-6, making each group contain 1×10⁵ CFU/mL of Escherichia coli, Salmonella enterica, Lactobacillus pentosus, or Lactobacillus reuteri. The mixture volume in each tube was 4 mL, and the tubes were placed in an incubator at a constant temperature of 37° C. The diluted Escherichia coli solution or the diluted Salmonella enterica solution were shaken at 120 rpm for 24 hours, forming Escherichia coli culture solutions or Salmonella enterica culture solutions of control group and test groups 6-1 to 6-6. The diluted Lactobacillus pentosus solution or the diluted Lactobacillus reuteri solution were cultured at 120 rpm for 24 hours, forming Lactobacillus pentosus culture solutions or Lactobacillus reuteri culture solutions of control group and test groups 6-1 to 6-6.

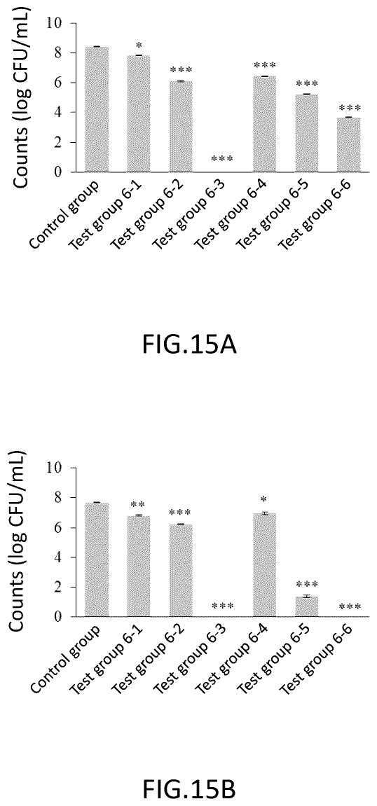

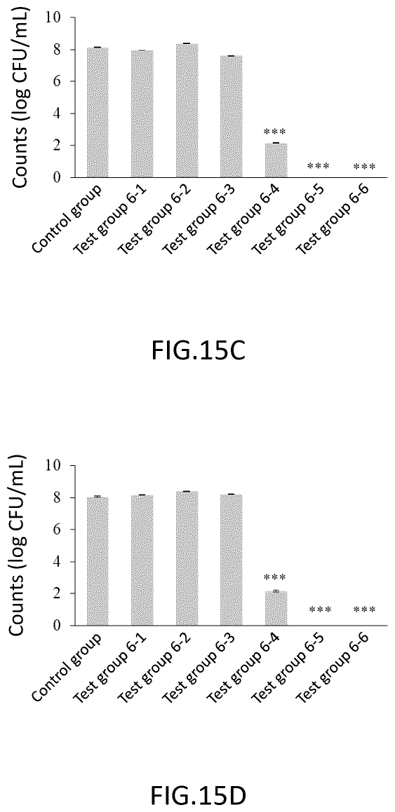

6 g of TSB medium or 11 g of MRSB and 3 g of agar were weighed, placed into a 500 mL serum bottle, and added with 200 mL of RO water. The serum bottle was then sterilized at 121° C. for 20 minutes to obtain a TSA medium or an MRS-agar (MRSA) medium. 1 mL of the Escherichia coli culture solutions, Salmonella enterica culture solutions, Lactobacillus pentosus culture solutions, or Lactobacillus reuteri culture solutions of each group were diluted using sequential 10-fold serial dilutions with 0.85% saline solution, and 1 mL of the diluted culture solutions at each gradient concentration were poured into the petri dishes. 20 mL of the TSA medium or the MRSA medium was added into each petri dish. After the liquid in the petri dishes was dried, the petri dishes were cultured at a constant temperature of 37° C. for 24 hours. The bacterial numbers in each petri dish were calculated based on the culture results. After multiplying back by the dilution factor, the calculated bacterial numbers of Escherichia coli in each group are shown in FIG. 15A, the calculated bacterial numbers of Salmonella enterica in each group are shown in FIG. 15B, the calculated bacterial numbers of Lactobacillus pentosus in each group are shown in FIG. 15C, and the calculated bacterial numbers of Lactobacillus reuteri in each group are shown in FIG. 15D.

In FIG. 15A to FIG. 15D, the ethanol extract of the fermented coffee composition can inhibit the growth of Escherichia coli and Salmonella enterica (test groups 6-1 to 6-3), but the anti-bacterial effect did not have an impact on beneficial bacteria such as Lactobacillus pentosus and Lactobacillus reuteri. On the other hand, the antibiotics indiscriminately damaged all types of bacteria (test groups 6-4 to 6-6) including beneficial bacteria. Therefore, compared to the antibiotics, the ethanol extract of the fermented coffee composition does not have negative impacts on the beneficial bacteria, displaying a targeted anti-bacterial effect.

### Test Example 7: Bacterial Cell Membrane Damage Assay

1.5 g of TSB medium was weighed, placed into a 100 mL Erlenmeyer flask, and added with 50 mL of RO water. The Erlenmeyer flask was then sterilized at 121° C. for 20 minutes and cooled to room temperature. Approximately 0.1 g of human Escherichia coli freeze-dried powder (purchased from the Food Industry Research and Development Institute, BCRC11509) or human Salmonella enterica freeze-dried powder (purchased from the Food Industry Research and Development Institute, BCRC15464) was added into the Erlenmeyer flask, and the Erlenmeyer flask was placed in an incubator at a constant temperature of 37° C. and shaken at a speed of 120 rpm for 24 hours to obtain an Escherichia coli solution or a Salmonella enterica solution. Test groups 7-1 to 7-3 were prepared using the ethanol extract of Preparation Example 1 (without any antibiotics), wherein concentrations of the ethanol extract were respectively 12.5 mg/mL, 25 mg/mL, and 50 mg/mL. 3 mL of the ethanol extract of Preparation Example 5 was used as a control group. Herein, the fermented coffee composition of Preparation Example 1 used for the test groups 7-1 to 7-3 was fermented for 5 days in the step (b).

The ethanol extracts of control group and test groups 7-1 to 7-3 were respectively placed into 4 tubes. The Escherichia coli solution or the Salmonella enterica solution was diluted with TSB medium, and 1 mL of the diluted Escherichia coli solution or the diluted Salmonella enterica solution was added respectively to the control group and the test groups 7-1 to 7-3, making each group contain 1×10⁸ CFU/mL of Escherichia coli or Salmonella enterica. The mixture volume in each tube was 4 mL, and the tubes were placed in an incubator at a constant temperature of 37° C. and shaken at 120 rpm for 24 hours, forming Escherichia coli culture solutions or Salmonella enterica culture solutions of control group and test groups 7-1 to 7-3. 1 mL of the Escherichia coli culture solutions or the Salmonella enterica culture solutions were centrifuged at 8000 rpm for 5 minutes to obtain supernatants and bacterial cells.

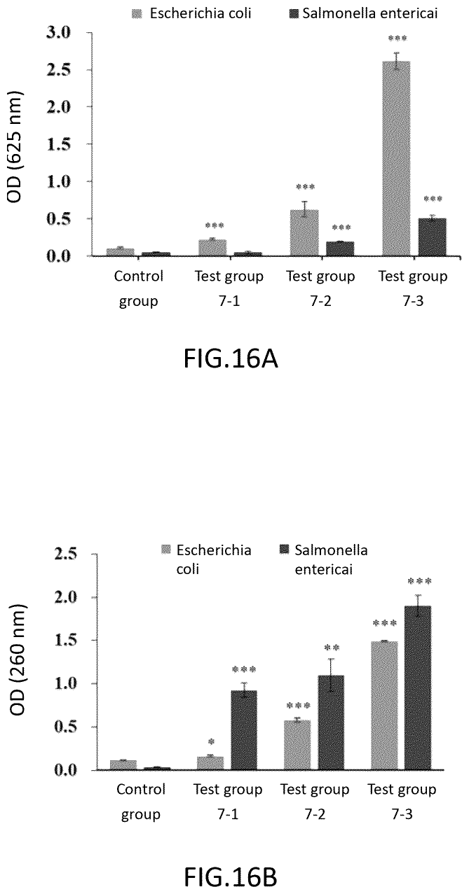

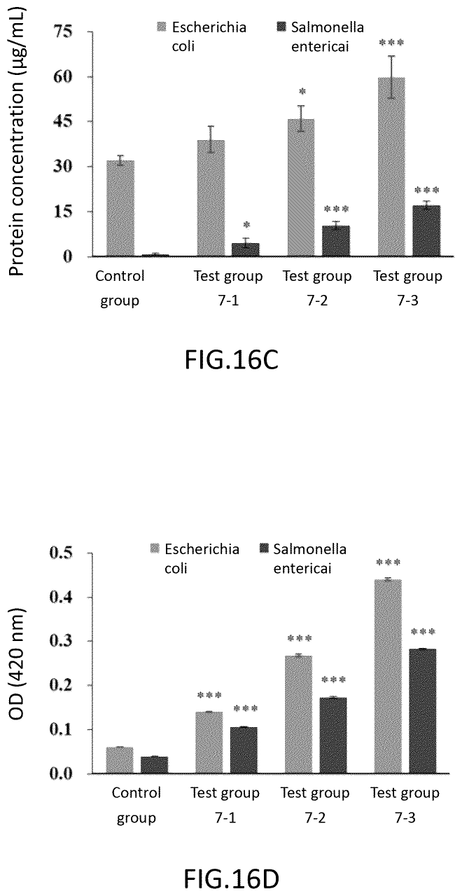

The bacterial cells were added with 1 mL of phosphate buffered saline (PBS) and centrifuged at 8000 rpm for 5 minutes. PBS was removed, and the washing process was repeated for 3 times. 0.5 mL of propidium iodide (PI) was added to the washed bacterial cells, and the washed bacterial cells were dispersed and placed in the dark for 15 minutes at room temperature for fluorescent staining. The stained bacterial cells were centrifuged at 8000 rpm for 5 minutes to remove propidium iodide, added with 1 mL of PBS, and centrifuged at 8000 rpm for 5 minutes. PBS was removed; and the washing process was repeated for 3 times. The washed bacterial cells were suspended in 1 mL of PBS and analyzed for intracellular fluorescence intensity using a fluorometer, with the excitation wavelength set to 495 nm and the emission wavelength set to 625 nm. The analyzed result is shown in FIG. 16A.

The supernatants were used for following analyses: (i) the nucleic acid concentration was analyzed by directly measuring the optical density (OD) at an ultraviolet (UV) wavelength of 260 nm; (ii) the protein concentration was determined after reacting the supernatants with Coomassie Blue for 5 minutes, followed by measuring the OD at 595 nm and calculating the protein concentration; and (iii) 950 μL of each supernatant was placed into a microcentrifuge tube, added with 50 μL of ortho-nitrophenyl-β-galactoside (ONPG) with a concentration of 1 mmol/L (mM), reacted at room temperature for 2 hours, and then transferred 200 μL reacted solution to a 96-well plate to measure the OD at 420 nm to analyze the β-galactosidase concentration. The results of (i) through (iii) were respectively shown in FIG. 16B to FIG. 16D.

The ethanol extracts of the fermented coffee composition of test groups 7-1 to 7-3 could damage the bacterial cell membrane, creating pores that allowed propidium iodide to penetrate and embed itself within the nucleic acids of the bacteria, then excited to emit red fluorescence, as shown in FIG. 16A. Once the cell membrane ruptured, DNA, RNA, and protein leaked out of the bacteria and could subsequently be collected from the supernatants after centrifugation, as shown in FIG. 16B and FIG. 16C. Furthermore, β-galactosidase residing in the bacterial cytoplasm was also released, preventing the bacteria from cleaving lactose into glucose and galactose and obstructing bacterial energy production, with the released β-galactosidase appearing yellow, as shown in FIG. 16D. According to the results in FIG. 16A to FIG. 16D, a higher concentration of the ethanol extract of the fermented coffee composition correlated with a greater degree of bacterial cell membrane damage, leading to the release of more intracellular substances. This demonstrates that the ethanol extract of the fermented coffee composition can effectively achieve an anti-bacterial effect by destroying the cell membrane.

### Test Example 8: Scanning Electron Microscope Analysis

1.5 g of TSB medium was weighed, placed into a 100 mL Erlenmeyer flask, and added with 50 mL of RO water. The Erlenmeyer flask was then sterilized at 121° C. for 20 minutes and cooled to room temperature. Approximately 0.1 g of human Escherichia coli freeze-dried powder (purchased from the Food Industry Research and Development Institute, BCRC11509) or human Salmonella enterica freeze-dried powder (purchased from the Food Industry Research and Development Institute, BCRC15464) was added into the Erlenmeyer flask, and the Erlenmeyer flask was placed in an incubator at a constant temperature of 37° C. and shaken at a speed of 120 rpm for 24 hours to obtain an Escherichia coli solution or a Salmonella enterica solution. Test groups 8-1 to 8-3 were prepared using the ethanol extract of Preparation Example 1 and cephalexin. 3 mL of the ethanol extract of Preparation Example 5 was used as a control group. 3 mL of the ethanol extract of Preparation Example 1 was used as the test groups 8-1 and 8-2 (without any antibiotics), wherein concentrations of the ethanol extract were respectively 25 mg/mL and 50 mg/mL. 3 mL of the antibiotic solution of Preparation Example 4 (an antibiotic solution of cephalexin, in which TSB medium was used for concentration adjustment to reach 30 μg/mL in the antibiotic solution) was used as the test group 8-3. Herein, the fermented coffee composition of Preparation Example 1 used for the test groups 8-1 to 8-3 was fermented for 5 days in the step (b).

The ethanol extracts or the antibiotic solution of control group and test groups 8-1 to 8-3 were respectively placed into 4 tubes. The Escherichia coli solution or the Salmonella enterica solution was diluted with TSB medium, and 1 mL of the diluted Escherichia coli solution or the diluted Salmonella enterica solution was added respectively to the control group and the test groups 8-1 to 8-3, making each group contain 1×10⁸ CFU/mL of Escherichia coli or Salmonella enterica. The mixture volume in each tube was 4 mL, and the tubes were placed in an incubator at a constant temperature of 37° C. and shaken at 120 rpm for 24 hours, forming Escherichia coli culture solutions or Salmonella enterica culture solutions of control group and test groups 8-1 to 8-3. 1 mL of the Escherichia coli culture solutions or the Salmonella enterica culture solutions were centrifuged at 8000 rpm for 5 minutes to obtain supernatants and bacterial cells.

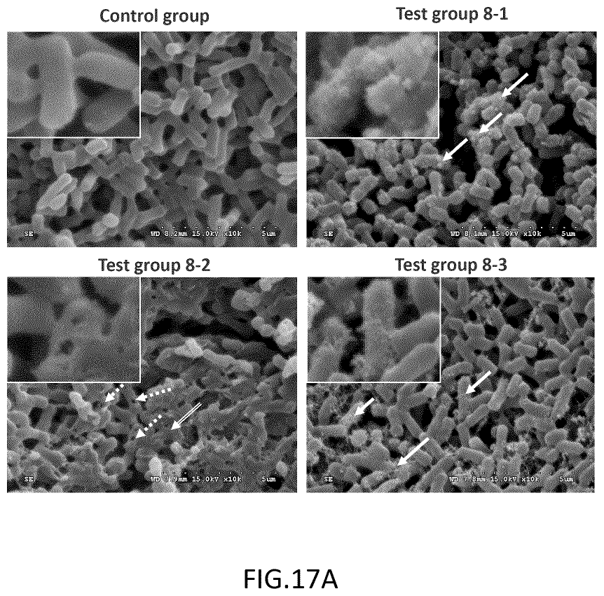

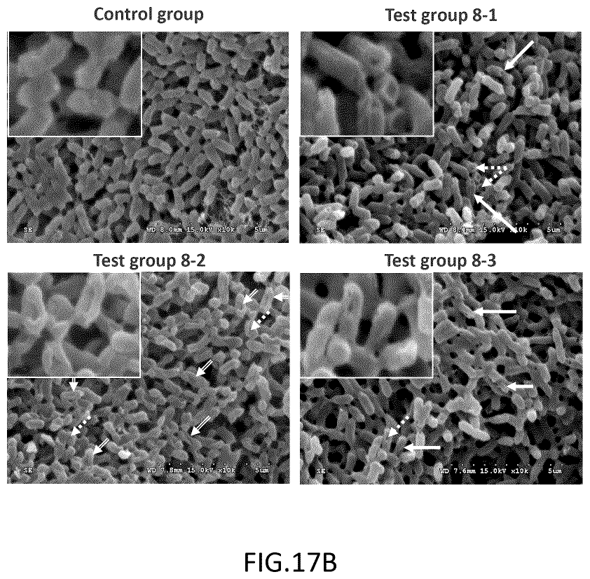

The bacterial cells were added with 1 mL of PBS and centrifuged at 8000 rpm for 5 minutes. PBS was removed, and the washing process was repeated for 3 times. The washed bacterial cells were suspended in 0.5 mL of PBS for fixing and dehydrated. After sputter-coated, the fixed bacterial cells were scanned with a scanning electron microscope (SEM). The appearance of the bacterial cells of Escherichia coli is shown in FIG. 17A, and the appearance of the bacterial cells of Salmonella enterica is shown in FIG. 17B.

According to the results in FIG. 17A and FIG. 17B, the ethanol extracts or the antibiotic solution of test groups 8-1 to 8-3 caused pores (indicated by white dashed arrows) to form in the cell membranes of Escherichia coli and Salmonella enterica. The released intracellular substances formed a mucous layer adhered to the outside of the cell membrane, making the bacterial surface rough (indicated by white solid arrows), and the bacterial cells became shrunken or even lysed (indicated by white double-lined arrows). Compared to using the antibiotic alone (test group 8-3), culturing the bacteria with 25 mg/mL of the ethanol extract (test group 8-1) induced external changes such as pore formation or rough surfaces more easily, and culturing with 50 mg/mL of the ethanol extract (test group 8-2) further promoted bacterial shrinkage or lysis, suggesting that the ethanol extract can achieve a better anti-bacterial effect than the antibiotic.

### Test Example 9: Electrophoretic Analysis

### 1. DNA Damage Assay

1.5 g of TSB medium was weighed, placed into a 100 mL Erlenmeyer flask, and added with 50 mL of RO water. The Erlenmeyer flask was then sterilized at 121° C. for 20 minutes and cooled to room temperature. Approximately 0.1 g of human Escherichia coli freeze-dried powder (purchased from the Food Industry Research and Development Institute, BCRC11509) or human Salmonella enterica freeze-dried powder (purchased from the Food Industry Research and Development Institute, BCRC15464) was added into the Erlenmeyer flask, and the Erlenmeyer flask was placed in an incubator at a constant temperature of 37° C. and shaken at a speed of 120 rpm for 24 hours to obtain an Escherichia coli solution or a Salmonella enterica solution. Test groups 9-1 to 9-3 were prepared using the ethanol extract of Preparation Example 1 (without any antibiotics), wherein concentrations of the ethanol extract were respectively 50 mg/mL, 25 mg/mL, and 12.5 mg/mL. 3 mL of the ethanol extract of Preparation Example 5 was used as a control group. Herein, the fermented coffee composition of Preparation Example 1 used for the test groups 9-1 to 9-3 was fermented for 5 days in the step (b).

The ethanol extracts of control group and test groups 9-1 to 9-3 were respectively placed into 4 tubes. The Escherichia coli solution or the Salmonella enterica solution was diluted with TSB medium, and 1 mL of the diluted Escherichia coli solution or the diluted Salmonella enterica solution was added respectively to the control group and the test groups 9-1 to 9-3, making each group contain 1×10⁸ CFU/mL of Escherichia coli or Salmonella enterica. The mixture volume in each tube was 4 mL, and the tubes were placed in an incubator at a constant temperature of 37° C. and shaken at 120 rpm for 24 hours, forming Escherichia coli culture solutions or Salmonella enterica culture solutions of control group and test groups 9-1 to 9-3. 1 mL of the Escherichia coli culture solutions or the Salmonella enterica culture solutions were centrifuged at 8000 rpm for 5 minutes to obtain supernatants and bacterial cells.

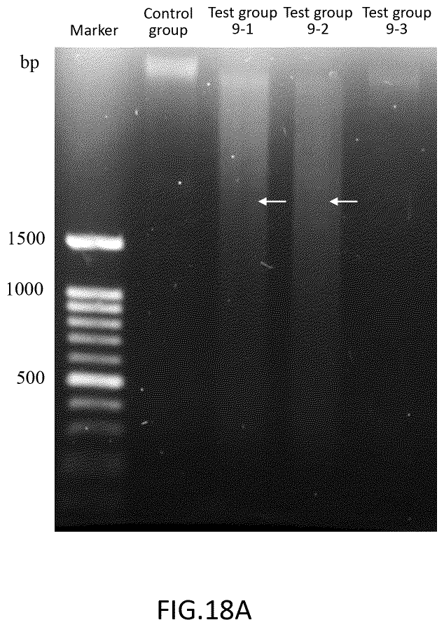

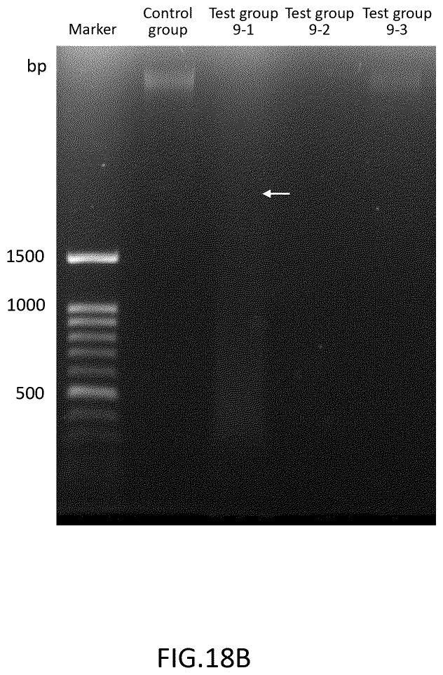

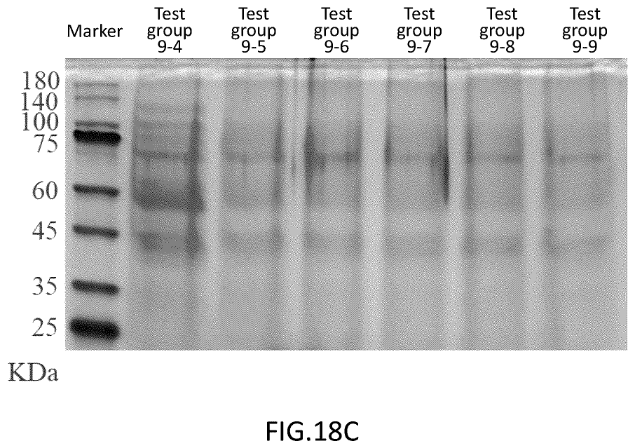

The bacterial cells were added with 1 mL of PBS and centrifuged at 8000 rpm for 5 minutes. PBS was removed, and the washing process was repeated for 3 times. The nucleic acids were isolated from the bacterial cells using the Blood/Cultured Cell Genomic DNA Extraction Mini Kit (ALPHAGEN APBGR200) to prepare samples of control group and test groups 9-1 to 9-3. 0.8 g of agarose was weighed, placed into a serum bottle, and added with 0.5× tris/borate/EDTA (TBE) buffer. The serum bottle was heated in a microwave oven to dissolve the agarose, forming a gel matrix. When the gel matrix was cooled to 60° C., 0.005% (v/v) of nucleic acid stain (HealthView Nucleic Acid Stain, Genomics) was added. The gel matrix was solidified after 25 minutes to 30 minutes to form a 0.8% gel. The gel was then transferred into an electrophoresis tank. The samples were thoroughly mixed with 6× sample loading dye (Genomics) and then loaded into wells along with the DNA marker (SIGMA). Electrophoresis was performed at 100 voltages (V) for 25 minutes to 30 minutes, and then images were captured using an UV transilluminator system. The electrophoresis results of Escherichia coli are shown in FIG. 18A, and the electrophoresis results of Salmonella enterica are shown in FIG. 18B.

According to the results in FIG. 18A and FIG. 18B, with the treatment of the ethanol extract of the fermented coffee composition, both Escherichia coli and Salmonella enterica exhibited smeared bands (indicated by white arrows) after electrophoresis, showing that structural changes occurred in the bacterial DNA. DNA damage further led to bacterial inactivation by altering metabolic function, and this trend became more pronounced as the concentration of the ethanol extract of the fermented coffee composition increased.

### 2. Protein Damage Assay

1.5 g of TSB medium was weighed, placed into a 100 mL Erlenmeyer flask, and added with 50 mL of RO water. The Erlenmeyer flask was then sterilized at 121° C. for 20 minutes and cooled to room temperature. Approximately 0.1 g of human Escherichia coli freeze-dried powder (purchased from the Food Industry Research and Development Institute, BCRC11509) was added into the Erlenmeyer flask, and the Erlenmeyer flask was placed in an incubator at a constant temperature of 37° C. and shaken at a speed of 120 rpm for 24 hours to obtain an Escherichia coli solution. Test groups 9-4 to 9-9 were prepared using 3 mL of the ethanol extract of Preparation Example 1 (without any antibiotics), wherein a concentration of the ethanol extract was 50 mg/mL, and the fermented coffee composition of Preparation Example 1 used for the test groups 9-4 to 9-9 was fermented for 5 days in the step (b).

The ethanol extracts of test groups 9-4 to 9-9 were respectively placed into 6 tubes. The Escherichia coli solution was diluted with TSB medium, and 1 mL of the diluted Escherichia coli solution was added respectively to the test groups 9-4 to 9-9. Each group contained 1×10⁸ CFU/mL of Escherichia coli and reacted for times shown in Table 9 below. The mixture volume in each tube was 4 mL, and the tubes were placed in an incubator at a constant temperature of 37° C. and shaken at 120 rpm for 24 hours, forming Escherichia coli culture solutions of test groups 9-4 to 9-9. 1 mL of the Escherichia coli culture solutions were centrifuged at 8000 rpm for 5 minutes.

TABLE 9
Reaction times of test groups 9-1 to 9-4 (T9-1 to T9-4)
with Escherichia coli in protein damage assay

| Ethanol extract | | | | | | |
|---|---|---|---|---|---|---|
| concentration of | Reaction time (hour) | | | | | |
| Preparation Example 1 | 0 | 3 | 6 | 15 | 18 | 24 |
| 50 mg/mL | T9-4 | T9-5 | T9-6 | T9-7 | T9-8 | T9-9 |

The bacterial cells were added with 1 mL of PBS and centrifuged at 8000 rpm for 5 minutes. PBS was removed, and the washing process was repeated for times. Total protein was extracted from the bacterial cells using the Minute™ protein extraction kit (Bio Pioneer Tech Co., LTD) to prepare samples of test groups 9-4 to 9-9. Sodium dodecyl sulfate polyacrylamide gel electrophoresis (SDS-PAGE) was prepared using a 4% stacking gel and a 10% resolving gel. The samples and the protein marker (SIGMA) were loaded into wells. Electrophoresis was performed at 100 voltages (V) for 120 minutes, and then images were captured using a camera. The electrophoresis results are shown in FIG. 18C.

According to the results in FIG. 18C, the longer the reaction time of the Escherichia coli with the ethanol extract of the fermented coffee composition, the lighter the color of the bands, indicating a reduction in protein expression. Furthermore, the bands of test groups 9-4 to 9-9 showed discontinuous gaps between 100 kDa and 140 kDa, suggesting that the ethanol extract of the fermented coffee composition can interfere with and inhibit protein synthesis of Escherichia coli, thereby affecting bacterial viability.

### Test Example 10: Reactive Oxygen Species Assay

1.5 g of TSB medium was weighed, placed into a 100 mL Erlenmeyer flask, and added with 50 mL of RO water. The Erlenmeyer flask was then sterilized at 121° C. for 20 minutes and cooled to room temperature. Approximately 0.1 g of human Escherichia coli freeze-dried powder (purchased from the Food Industry Research and Development Institute, BCRC11509) was added into the Erlenmeyer flask, and the Erlenmeyer flask was placed in an incubator at a constant temperature of 37° C. and shaken at a speed of 120 rpm for 24 hours to obtain an Escherichia coli solution. Test groups 10-1 to 10-6 were prepared using the ethanol extract of Preparation Example 1, and test groups 10-7 to 10-9 were prepared using the ethanol extract of Preparation Example 3 (without any antibiotics), wherein a concentration of the ethanol extract of test groups 10-1 to 10-9 was 50 mg/mL. The ethanol extract of Preparation Example 5 was used as a control group. The ethanol extracts of test groups 10-1 to 10-9 were treated with catalase or peroxidase according to the conditions shown in Table 10 to prepare 3 mL of reaction solutions. The purpose of incubating in a 95° C. water bath for 5 minutes in some test groups was to inactivate catalase or peroxidase.

TABLE 10
Conditions for preparing reaction solutions of test groups
10-1 to 10-9 (T10-1 to T10-9) in Test Example 10

| Groups | Enzyme for treatment | Treatment |
|---|---|---|
| T10-1 | No | No treatment. |
| T10-2 | No | Incubating in a 95° C. water bath |
| | | for 5 minutes. |
| T10-3 | 200 μg/mL of catalase | Placing for 2 hours. |
| T10-4 | 200 μg/mL of catalase | Placing for 2 hours and incubating |
| | | in a 95° C. water bath for 5 minutes. |
| T10-5 | 200 μg/mL of peroxidase | Placing for 2 hours. |
| T10-6 | 200 μg/mL of peroxidase | Placing for 2 hours and incubating |
| | | in a 95° C. water bath for 5 minutes. |
| T10-7 | No | No treatment. |
| T10-8 | 200 μg/mL of peroxidase | Placing for 2 hours. |
| T10-9 | 200 μg/mL of catalase | Placing for 2 hours. |

After the reaction solutions of each group were cooled to room temperature, the reaction solutions of control group and test groups 10-1 to 10-9 were respectively placed into 10 tubes. The Escherichia coli solution was diluted with TSB medium, and 1 mL of the diluted Escherichia coli solution was added respectively to the control group and the test groups 10-1 to 10-9, making each group contain 1×10⁷ CFU/mL to 1×10⁸ CFU/mL of Escherichia coli. The mixture volume in each tube was 4 mL, and the tubes were placed in an incubator at a constant temperature of 37° C. and shaken at 120 rpm for 18 hours, forming Escherichia coli culture solutions of control group and test groups 10-1 to 10-9.

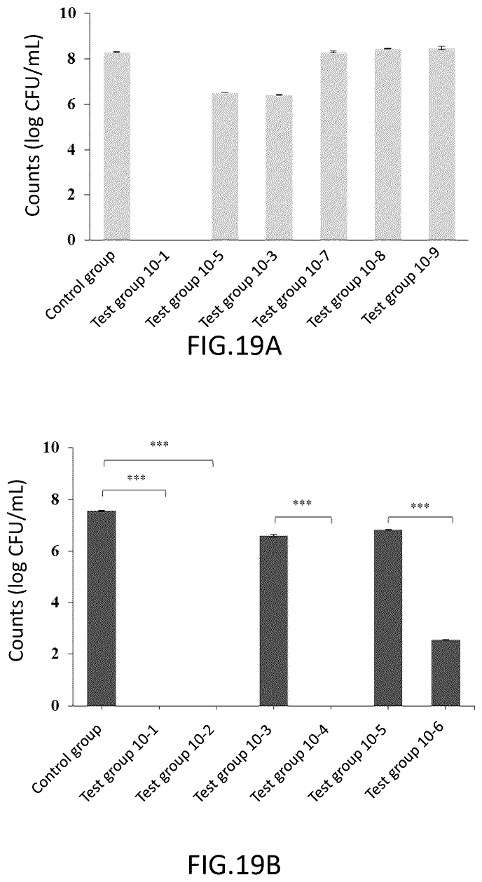

1 mL of the Escherichia coli culture solutions of each group were diluted using sequential 10-fold serial dilutions with 0.85% saline solution, and 1 mL of the diluted culture solutions at each gradient concentration were poured into the petri dishes. 20 mL of the TSA medium was added into each petri dish. After the liquid in the petri dishes was dried, the petri dishes were cultured at a constant temperature of 37° C. for 24 hours. The bacterial numbers in each petri dish were calculated based on the culture results. After multiplying back by the dilution factor, the calculated bacterial numbers of Escherichia coli in each group are shown in FIG. 19A and FIG. 19B.

According to the results in FIG. 19A and FIG. 19B, the ethanol extract of the unfermented coffee composition did not possess an anti-bacterial ability (test groups 10-7 to 10-9). The anti-bacterial effect of the fermented coffee composition was eliminated after the treatment of peroxidase or catalase (test groups 10-3 and 10-5) but could remain active when catalase or peroxidase was inactivated by hot water bath treatment (test groups 10-4 and 10-6). Peroxidase or catalase can prevent the excessive accumulation of reactive oxygen species (ROS) within cells, protecting the cells from oxidative damage. Therefore, the anti-bacterial effect of the ethanol extract of the fermented coffee composition is achieved by causing an imbalance in the internal redox state of the bacteria, leading to a pro-oxidative force greater than the anti-oxidative force. As a result, the oxidative stress resulting from the accumulation of ROS inactivates the bacteria.

### Test Example 11: Cell Inflammatory Response Assay

Porcine intestinal epithelial cells IPEC-J2 and mouse macrophages RAW264.7 were seeded in 6-well plates, each containing 2 mL of complete medium. The complete medium was Dulbecco's Modified Eagle Medium (DMEM) (SH30243, HyClone) containing 10 vol % of fetal bovine serum (FBS) and 1 vol % of triple antibiotic (Penicillin/Streptomycin/Amphotericin B, PSA). When the cells were completely adhered to the bottom of each 6-well plate, the complete medium was replaced with 2 mL of serum-free DMEM. The cells were divided into a negative control group, positive control groups 1 to 3, and test groups 11-1 to 11-9. Lipopolysaccharide (LPS) and the ethanol extract of Preparation Example 1 were added to the cells of each group according to Table 11 to achieve concentrations of LPS and the ethanol extract in the cell culture media of each group shown in Table 11. Herein, the fermented coffee composition of Preparation Example 1 used for the test groups 11-1 to 11-9 was fermented for 5 days in the step (b).

TABLE 11
Concentrations of LPS and ethanol extract in the cell
culture media of negative control group (N1), positive
control groups 1 to 3 (P1 to P3), and test groups
11-1 to 11-9 (T11-1 to T11-9) in Test Example 11

| | LPS | | Ethanol extract concentration of the fermented | |
|---|---|---|---|---|
| Groups | concentration | | coffee composition of Preparation Example 1 | |
| N1 | 0 | | 0 | |
| P1 | 10 | μg/mL | 0 | |
| P2 | 4 | μg/mL | 0 | |
| P3 | 40 | μg/mL | 0 | |
| T11-1 | 10 | μg/mL | 50 | μg/mL |
| T11-2 | 10 | μg/mL | 100 | μg/mL |
| T11-3 | 10 | μg/mL | 200 | μg/mL |
| T11-4 | 4 | μg/mL | 50 | μg/mL |
| T11-5 | 4 | μg/mL | 100 | μg/mL |
| T11-6 | 4 | μg/mL | 200 | μg/mL |
| T11-7 | 40 | μg/mL | 50 | μg/mL |
| T11-8 | 40 | μg/mL | 100 | μg/mL |
| T11-9 | 40 | μg/mL | 200 | μg/mL |

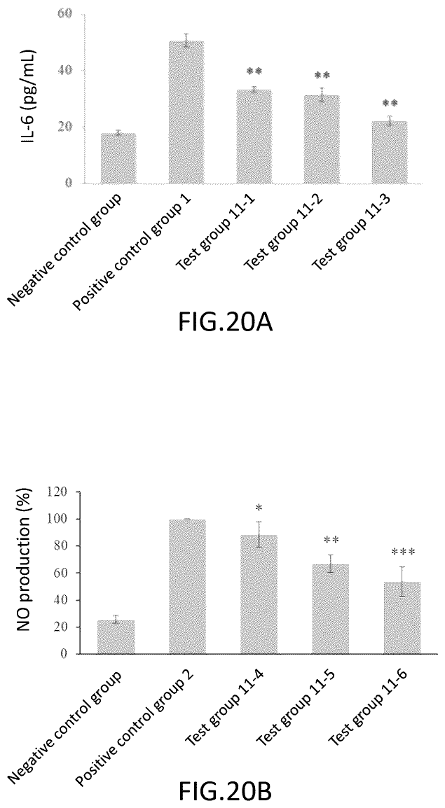

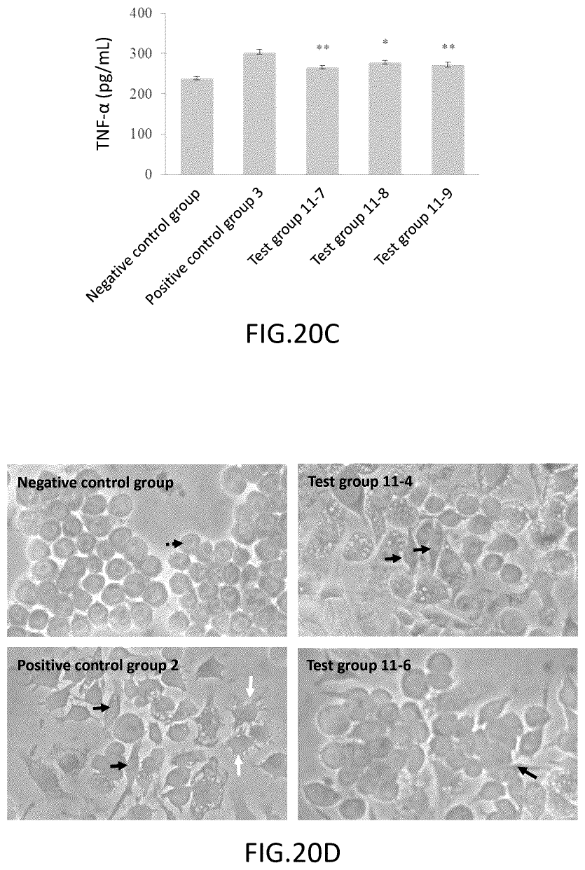

The cells were cultured in an incubator for 1 hour (positive control group 1, test groups 11-1 to 11-3), 15 hours (positive control group 3, test groups 11-7 to 11-9), or 24 hours (positive control group 2, test groups 11-4 to 11-6), and supernatants (culture medium) of each group were collected. The concentrations of IL-6 protein were measured using the Pig IL-6 ELISA Kit (MBS2701081, MyBioSource), the NO production rates were determined by Griess assay, and the concentrations of TNF-α protein were measured using the Porcine TNF-α ELISA Kit (MBS2701342, MyBioSource). The production of the three inflammatory factors increased differently after the induction by LPS, and thus the induction times varied. The results of IL-6 concentrations using IPEC-J2 cells are shown in FIG. 20A, the results of NO production rates using RAW264.7 cells are shown in FIG. 20B, and the results of TNF-α concentrations using IPEC-J2 cells are shown in FIG. 20C.

The RAW264.7 cells of negative control group, positive control group 2, and test groups 14-4 and 14-6 after culturing were observed under a microscope at 200× magnification. The observed cell morphology is shown in FIG. 20D.

According to the results in FIG. 20A to FIG. 20C, the inflammatory response induced by LPS increased the expression of IL-6 and TNF-α in IPEC-J2 cells and promoted NO production in macrophages. The ethanol extract of the fermented coffee composition was able to reduce IL-6 concentration (test groups 14-1 to 14-3) and TNF-α concentration (test groups 14-7 to 14-9), and decrease the NO production rate (test groups 14-4 to 14-6). Furthermore, as the concentration of the ethanol extract of the fermented coffee composition increased, the decrease in IL-6 concentration and NO production rate became more significant. According to the results in FIG. 20D, normal RAW264.7 cells appeared spherical (indicated by a black dashed arrow), while cells developed pseudopod-like shapes (indicated by white arrows) or spread outwards (indicated by black solid arrows) after the induction of an inflammatory response. Cells treated with the ethanol extract of the fermented coffee composition did not form pseudopods. This shows that the ethanol extract of the fermented coffee composition can alleviate the inflammatory response induced by LPS.

### Test Example 12: Bacterial Inflammatory Response Assay

Mouse macrophages RAW264.7 were cultured in 96-well plates, each containing 100 μL of complete medium. The complete medium was DMEM (SH30243, HyClone) containing 10 vol % of FBS and 1 vol % of PSA. When the cells were completely adhered to the bottom of each 96-well plate, the complete medium was replaced with 100 μL of serum-free DMEM. The cells were divided into a control group and test groups 12-1 to 12-6.

1.5 g of TSB medium was weighed, placed into a 100 mL Erlenmeyer flask, and added with 50 mL of RO water. The Erlenmeyer flask was then sterilized at 121° C. for 20 minutes and cooled to room temperature. Approximately 0.1 g of human Escherichia coli freeze-dried powder (purchased from the Food Industry Research and Development Institute, BCRC11509) was added into the Erlenmeyer flask, and the Erlenmeyer flask was placed in an incubator at a constant temperature of 37° C. and shaken at a speed of 120 rpm for 24 hours to obtain an Escherichia coli solution. According to Table 12 below, bacterial culture media of test groups 12-1 to 12-6 were prepared using the ethanol extract of Preparation Example 1 and antibiotics (amoxicillin (AMX) or amoxicillin-clavulanic acid (AMX-CA)). The ethanol extract of Preparation Example 5 was added in the control group. The ethanol extract of Preparation Example 1 was added in the test group 12-2. The antibiotic solution of Preparation Example 4 (an antibiotic solution of amoxicillin or amoxicillin-clavulanic acid, in which TSB medium was used for concentration adjustment) was added in the test groups 12-3 and 12-5. The anti-bacterial composition of Example 1 (concentrations and antibiotic types as shown in Table 12) was added in the test groups 12-4 and 12-6. Herein, the fermented coffee composition of Preparation Example 1 used for the test groups 12-2, 12-4, and 12-6 was fermented for 5 days in the step (b).

TABLE 12
Ethanol extract concentrations, antibiotic concentrations, and
antibiotic types in bacterial culture media of test groups 12-1
to 12-6 (T12-1 to T12-6) and control group in Test Example 12

| | Ethanol extract concentration | | |
|---|---|---|---|
| | of the fermented coffee | | |
| | composition of Preparation | Antibiotic | Antibiotic |
| Groups | Example 1 | concentration | type |
| T12-1 | 0 | 0 | N/A |
| T12-2 | 6 mg/mL | 0 | N/A |
| T12-3 | 0 | 1000 μg/mL | AMX-CA |
| T12-4 | 6 mg/mL | 1000 μg/mL | AMX-CA |
| T12-5 | 0 | 1000 μg/mL | AMX |
| T12-6 | 6 mg/mL | 1000 μg/mL | AMX |
| Control | Ethanol extract of Example 5 | 0 | N/A |
| group | | | |

The bacterial culture media of control group and test groups 12-1 to 12-6 were respectively placed into 7 tubes. The Escherichia coli solution was diluted with TSB medium, and 1 mL of the diluted Escherichia coli solution was added respectively to the control group and the test groups 12-1 to 12-6, making each group contain 1×10⁸ CFU/mL of Escherichia coli. The mixture volume in each tube was 4 mL, and the tubes were placed in an incubator at a constant temperature of 37° C. and shaken at 120 rpm for 24 hours, forming Escherichia coli culture solutions of control group and test groups 12-1 to 12-6.

The Escherichia coli culture solutions of control group and test groups 12-1 to 12-6 were centrifuged at 8000 rpm for 5 minutes to remove bacterial cells and collect supernatants. The supernatants of each group were added to the 96-well plates containing RAW264.7. The macrophages were then cultured in an incubator for 24 hours, and NO production rates were measured from supernatants (culture medium) of each group.

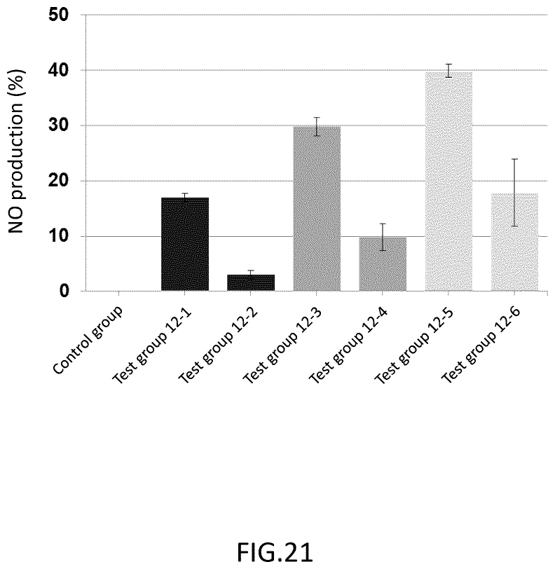

Antibiotics inhibit the synthesis of the Escherichia coli cell wall, causing the bacteria cells to lyse and release LPS, which induces an inflammatory response that promotes NO production in macrophages. According to the results in FIG. 21, because the test group 12-2 was treated with the ethanol extract of the fermented coffee composition, it showed a lower NO production rate compared to the test group 12-1. Furthermore, when the ethanol extract of the fermented coffee composition was used simultaneously with antibiotics in test groups 12-4 and 12-6, the NO production rate was also significantly reduced compared to the test groups 12-3 and 12-5 using the corresponding antibiotics alone. This suggests that the ethanol extract of the fermented coffee composition can alleviate the inflammatory response induced by the release of LPS caused by the antibiotics.

In summary, the anti-bacterial composition of the present invention comprises a coffee composition extracted with alcohol or water and an antibiotic. The extract of the coffee composition can alleviate the inflammatory response induced by LPS through reducing the production of proteins such as IL-6 and TNF-α in cells and the production of NO in macrophages. The anti-bacterial synergistic effect formed by the combination of the ethanol extract and the antibiotics allows the anti-bacterial composition of the present invention to exhibit a better anti-bacterial effect than using the antibiotic alone. Besides, the anti-bacterial effect is also active against drug-resistant bacteria, which overcomes the problem of decreased anti-bacterial ability associated with the increasing frequency of use of traditional anti-bacterial medicaments.

What is claimed is:

1. An anti-bacterial composition comprising:
an extract of a coffee composition, in which the coffee composition comprises 92 wt % to 95 wt % of a solid content and 5 wt % to 8 wt % of water; the solid content comprises 20 wt % to 70 wt % of coffee beans, and 30 wt % to 80 wt % of an auxiliary material; and the coffee composition has a carbon-nitrogen ratio ranging from 35 to 50; and
an antibiotic selected from penicillins, cephalosporins, fluoroquinolones, tetracyclines, chloramphenicols, aminoglycosides, and a combination thereof;
wherein, the ratio of the coffee composition to the antibiotic is in an amount ranging from 1.5:1 to 25000:1; and the extract of the coffee composition comprises a water extract of the coffee composition, an alcohol extract of the coffee composition, or a combination thereof.

2. The anti-bacterial composition as claimed in claim 1, wherein the extract of the coffee composition comprises the alcohol extract of the coffee composition.

3. The anti-bacterial composition as claimed in claim 1, wherein the coffee beans are raw coffee beans, roasted coffee beans, or recycled coffee grounds.

4. The anti-bacterial composition as claimed in claim 3, wherein the raw coffee beans and the roasted coffee beans are crushed or ground.

5. The anti-bacterial composition as claimed in claim 1, wherein the alcohol extract is prepared by extracting the coffee composition with an ethanol aqueous solution having a concentration of 60% to 80%; and, the ratio of the coffee composition to the ethanol aqueous solution is from 1 gram to 3 mL to 1 gram to 7 mL.

6. The anti-bacterial composition as claimed in claim 1, wherein the auxiliary material comprises a corn grit, a beet grit, a rice husk, a shelled soybean meal, a broken rice, or a combination thereof.

7. The anti-bacterial composition as claimed in claim 1, wherein the coffee composition is unfermented or fermented by Aspergillus oryzae.

8. The anti-bacterial composition as claimed in claim 1, wherein the coffee composition is prepared by the following processes: (1) mixing 40 wt % to 60 wt % of the solid content and 40 wt % to 60 wt % of water to obtain a mixture; (2) heating the mixture at 115° C. to 125° C. under a pressure of 1.0 bar to 1.5 bars for 20 minutes to 60 minutes to obtain an autoclaved mixture; and (3) cooling and drying the autoclaved mixture until the water content of the autoclaved mixture is reduced to 5 wt % to 8 wt % to obtain the coffee composition.

9. The anti-bacterial composition as claimed in claim 8, wherein the coffee composition is further fermented by Aspergillus oryzae by inoculating Aspergillus oryzae in the coffee composition with 1×10⁷ to 1×10⁸ spores per gram of coffee composition, and fermenting at 25° C. to 35° C. for 3 days to 7 days.

10. The anti-bacterial composition as claimed in claim 1, wherein the penicillin is selected from penicillin G, penicillin V, ampicillin, amoxicillin, and amoxicillin-clavulanic acid.

11. The anti-bacterial composition as claimed in claim 1, wherein the cephalosporin is selected from cephalexin, cephadroxil, and ceftiofur.

12. The anti-bacterial composition as claimed in claim 1, wherein the fluoroquinolone is selected from enrofloxacin, ofloxacin, danofloxacin, ciprofloxacin, and norfloxacin.

13. The anti-bacterial composition as claimed in claim 1, wherein the tetracycline is selected from oxytetracycline, chlortetracycline, and doxycycline.

14. The anti-bacterial composition as claimed in claim 1, wherein the chloramphenicol is selected from chloramphenicol, thiamphenicol, and florfenicol.

15. The anti-bacterial composition as claimed in claim 1, wherein the aminoglycoside is selected from streptomycin, neomycin, kanamycin, gentamicin, spectinomycin, and apramycin.

16. The anti-bacterial composition as claimed in claim 1, wherein the anti-bacterial composition has bacterial inhibition effect on a bacterium selected from Salmonella spp., Escherichia spp., Pasteurella spp., Streptococcus spp., Gallibacterium spp., Staphylococcus spp., Riemerella spp., Pseudomonas spp., and Klebsiella spp.

17. The anti-bacterial composition as claimed in claim 16, wherein the bacterium is selected from Salmonella enterica, Salmonella choleraesuis, Escherichia coli, Pasteurella multocida, Glaesserella parasuis, Streptococcus suis, Gallibacterium anatis, Staphylococcus hyicus, Riemerella anatipestifer, Pseudomonas aeruginosa, and Klebsiella pneumoniae.

18. The anti-bacterial composition as claimed in claim 16, wherein the bacterium is a drug-resistant bacterium.

19. The anti-bacterial composition as claimed in claim 17, wherein the bacterium is a drug-resistant bacterium.

20. A method for treating bacterial infection, comprising administering to an animal in need thereof the anti-bacterial composition as claimed in claim 1.

21. The method as claimed in claim 20, wherein the animal is pig, cattle, cow, sheep, goat, chicken, duck, goose, fish, or shrimp.
